(19) United States
(12) Patent Application Publication
(10) Pub. No.: US 2006/0019948 A1
(43) Pub. Date: Jan. 26, 2006

(54) Methylidene-D-xylopyranosyl- and oxo-D-xylopyranosyl-substituted phenyl derivatives, medicaments containing such compounds, their use and process for their manufacture

(75) Inventors: Matthias Eckhardt, Biberach (DE); Frank Himmelsbach, Mittelbiberach (DE); Peter Eickelmann, Mittelbiberach (DE); Leo Thomas, Biberach (DE); Edward Leon Barsoumian, Osaka (JP)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/182,986

(22) Filed: Jul. 15, 2005

(30) Foreign Application Priority Data
Jul. 17, 2004 (DE) 102004034690

(51) Int. Cl. A61K 31/5377; A61K 31/496; A61K 31/452; A61K 31/4025; A61K 31/366; C07D 413/02; C07D 405/02

(52) U.S. Cl. 514/231.5; 514/254.1; 514/326; 514/422; 514/460; 544/149; 548/517; 544/374; 546/214

(57) ABSTRACT

D-Xylopyranosyl-substituted phenyls of general formula I 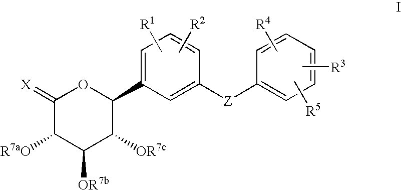
wherein the groups R¹ to R⁵, X, Z and R^{7a}, R^{7b}, R^{7c} are defined as in claim 1, have an inhibiting effect on the sodium-dependent glucose cotransporter SGLT. The present invention also relates to pharmaceutical compositions for the treatment of metabolic disorders.

Benefit of DE 102004034690 filed Jul. 17, 2004 is hereby claimed and said applications are herein incorporated by reference.

The present invention relates to D-xylopyranosyl-substituted phenyls of general formula I 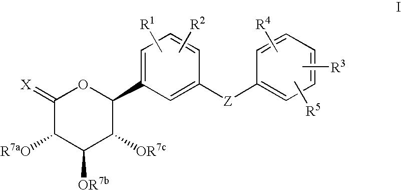
wherein the groups R¹ to R⁵, X, Z and R^{7a}, R^{7b}, R^{7c} are as hereinbefore defined, including the tautomers, the stereoisomers, the mixtures thereof and the salts thereof. The invention further relates to pharmaceutical compositions containing a compound of formula I according to the invention as well as the use of a compound according to the invention for preparing a pharmaceutical composition for the treatment of metabolic disorders. The invention also relates to processes for preparing a pharmaceutical composition and a compound according to the invention.

Compounds which have an inhibitory effect on the sodium-dependent glucose cotransporter SGLT are proposed in the literature for the treatment of diseases, particularly diabetes.

Glucopyranosyl-substituted aromatic groups and the preparation thereof and their possible activity as SGLT2 inhibitors are known from published International Patent Applications WO 98/31697, WO 01/27128, WO 02/083066, WO 03/099836, WO 04/13118, WO 04/80990, WO 04/52902, WO 04/52903 and WO 05/12326.

### AIM OF THE INVENTION

The aim of the present invention is to indicate new pyranosyl-substituted phenyls, particularly those which have an effect on sodium-dependent glucose cotransporter SGLT, particularly SGLT2. A further aim of the present invention is to indicate pyranosyl-substituted phenyls which, by comparison with known structurally similar compounds, have a greater inhibitory effect on the sodium-dependent glucose cotransporter SGLT2 in vitro and/or in vivo and/or have improved pharmacological or pharmacokinetic properties.

Moreover the present invention also sets out to prepare new pharmaceutical compositions which are suitable for the prevention and/or treatment of metabolic disorders, particularly diabetes.

The invention also relates to a process for preparing the compounds according to the invention.

Further aims of the present invention will immediately become apparent to the skilled man from the remarks above and hereinafter.

### OBJECT OF THE INVENTION

In a first aspect the invention relates to D-xylopyranosyl-substituted phenyls of general formula I 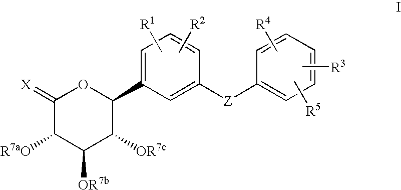
wherein
R¹ denotes hydrogen, fluorine, chlorine, bromine, C₁₋₆-alkyl, C₂₋₆-alkynyl, C₂₋₆-alkenyl, C₃₋₇-cycloalkyl, C₃₋₇-cycloalkyl-C₁₋₃-alkyl, C₅₋₇-cycloalkenyl, C₅₋₇-cycloalkenyl-C₁₋₃-alkyl, C₁₋₄-alkylcarbonyl, arylcarbonyl, heteroarylcarbonyl, aminocarbonyl, C₁₋₄-alkylaminocarbonyl, di-(C₁₋₃-alkyl)aminocarbonyl, pyrrolidin-1-ylcarbonyl, piperidin-1-ylcarbonyl, morpholin-4-ylcarbonyl, piperazin-1-ylcarbonyl, 4-(C₁₋₄-alkyl)piperazin-1-ylcarbonyl, C₁₋₄-alkoxycarbonyl, amino, C₁₋₄-alkylamino, di-(C₁₋₃-alkyl)amino, pyrrolidin-1-yl, piperidin-1-yl, morpholin-4-yl, piperazin-1-yl, 4-(C₁₋₄-alkyl)piperazin-1-yl, C₁₋₄-alkylcarbonylamino, C₁₋₆-alkyloxy, C₃₋₇-cycloalkyloxy, C₅₋₇-cycloalkenyloxy, aryloxy, C₁₋₄-alkylsulphanyl, C₁₋₄-alkylsulphinyl, C₁₋₄-alkylsulphonyl, C₃₋₇-cycloalkylsulphanyl, C₃₋₇-cycloalkylsulphinyl, C₃₋₇-cycloalkylsulphonyl, C₅₋₇-cycloalkenylsulphanyl, C₅₋₇-cycloalkenylsulphinyl, C₅₋₇-cycloalkenylsulphonyl, arylsulphanyl, arylsulphinyl, arylsulphonyl, hydroxy, cyano or nitro,
   while alkyl, alkenyl, alkynyl, cycloalkyl and cycloalkenyl groups may be partly or completely fluorinated or may be mono- or disubstituted by identical or different substituents selected from chlorine, hydroxy, C₁₋₃-alkoxy and C₁₋₃-alkyl, and
   in cycloalkyl and cycloalkenyl groups one or two methylene groups may be replaced independently of one another by O, S, CO, SO or SO₂, and
   in N-heterocycloalkyl groups a methylene group may be replaced by CO or SO₂, and
R² denotes hydrogen, fluorine, chlorine, bromine, hydroxy, C₁₋₄-alkyl, C₁₋₄-alkoxy, cyano or nitro, while alkyl groups may be mono- or polysubstituted by fluorine, or
   in the event that R¹ and R² are bound to two C atoms of the phenyl ring which are adjacent to one another, R¹ and R² may be joined together in such a way that R¹ and R² together form a C₃₋₅-alkylene or C₃₋₅-alkenylene bridge, which may be partly or totally fluorinated or mono- or disubstituted by identical or different substituents selected from chlorine, hydroxy, C₁₋₃-alkoxy and C₁₋₃-alkyl and wherein one or two methylene groups may be replaced independently of one another by O, S, CO, SO, SO₂ or NR^{N},
R³ denotes hydrogen, fluorine, chlorine, bromine, C₁₋₆-alkyl, C₂₋₆-alkynyl, C₂₋₆-alkenyl, C₃₋₇-cycloalkyl, C₃₋₇-cycloalkyl-C₁₋₃-alkyl, C₅₋₇-cycloalkenyl, C₅₋₇-cycloalkenyl-C₁₋₃-alkyl, aryl, heteroaryl, C₁₋₄-alkylcarbonyl, arylcarbonyl, heteroarylcarbonyl, aminocarbonyl, C₁₋₄-alkylaminocarbonyl, di-(C₁₋₃-alkyl)aminocarbonyl, pyrrolidin-1-ylcarbonyl, piperidin-1-ylcarbonyl, morpholin-4-ylcarbonyl, piperazin-1-ylcarbonyl, 4-(C₁₋₄-alkyl)piperazin-1-ylcarbonyl, hydroxycarbonyl, C₁₋₄-alkoxycarbonyl, C₁₋₄-alkylamino, di-(C₁₋₃-alkyl)amino, pyrrolidin-1-yl, piperidin-1-yl, morpholin-4-yl, piperazin-1-yl, 4-(C₁₋₄-alkyl)piperazin-1-yl, C₁₋₄-alkylcarbonylamino, arylcarbonylamino, heteroarylcarbonylamino, C₁₋₄-alkylsulphonylamino, arylsulphonylamino, C₁₋₆-alkoxy, C₃₋₇-cycloalkyloxy, C₅₋₇-cycloalkenyloxy, aryloxy, heteroaryloxy, C₁₋₄-alkylsulphanyl, C₁₋₄-alkylsulphinyl, C₁₋₄-alkylsulphonyl, C₃₋₇-cycloalkyl-sulphanyl, C₃₋₇-cycloalkylsulphinyl, C₃₋₇-cycloalkylsulphonyl, C₅₋₇-cycloalkenylsulphanyl, C₅₋₇-cycloalkenylsulphinyl, C₅₋₇-cycloalkenylsulphonyl, arylsulphanyl, arylsulphinyl, arylsulphonyl, amino, hydroxy, cyano or nitro,
   while alkyl, alkenyl, alkynyl, cycloalkyl and cycloalkenyl groups may be partly or totally fluorinated or mono- or disubstituted by identical or different substituents selected from chlorine, hydroxy, C₁₋₃-alkoxy and C₁₋₃-alkyl, and
   in cycloalkyl and cycloalkenyl groups one or two methylene groups may be replaced independently of one another by O, S, CO, SO or SO₂, and
   in N-heterocycloalkyl groups a methylene group may be replaced by CO or SO₂, and
R⁴ denotes hydrogen, fluorine, chlorine, bromine, iodine, cyano, nitro, C₁₋₃-alkyl, C₁₋₃-alkoxy or methyl or methoxy substituted by 1 to 3 fluorine atoms, or
   in the event that R³ and R⁴ are bound to two C atoms of the phenyl ring which are adjacent to one another, R³ and R⁴ may be joined together in such a way that R³ and R⁴ together form a C₃₋₅-alkylene or C₃₋₅-alkenylene bridge, which may be partly or totally fluorinated or mono- or disubstituted by identical or different substituents selected from chlorine, hydroxy, C₁₋₃-alkoxy and C₁₋₃-alkyl and wherein one or two methylene groups may be replaced independently of one another by O, S, CO, SO, SO₂ or NR^{N},
R⁵ denotes hydrogen, fluorine, chlorine, bromine, iodine, cyano, nitro, C₁₋₃-alkyl, C₁₋₃-alkoxy or methyl or methoxy substituted by 1 to 3 fluorine atoms, and
R^{N} independently of one another denote H or C₁₋₄-alkyl,
L are selected independently of one another from among fluorine, chlorine, bromine, iodine, C₁₋₃-alkyl, difluoromethyl, trifluoromethyl, C₁₋₃-alkoxy, difluoromethoxy, trifluoromethoxy and cyano,
R^{7a}, R^{7b},
R^{7c} independently of one another have a meaning selected from among hydrogen, (C₁₋₁₈-alkyl)carbonyl, (C₁₋₁₈-alkyl)oxycarbonyl, arylcarbonyl and aryl-(C₁₋₃-alkyl)-carbonyl,
X denotes oxygen, or
   methylidene, fluoromethylidene, C₁₋₆-alkyl-methylidene, C₂₋₆-alkenyl-methylidene, C₂₋₆-alkynyl-methylidene, C₃₋₇-cycloalkyl-methylidene, C₅₋₇cycloalkenyl-methylidene, C₃₋₇-cycloalkylidene, C₅₋₇-cycloalkenylidene, C₃₋₇-cycloalkyl-C₁₋₃-alkyl-methylidene, C₅₋₇-cycloalkenyl-C₁₋₃-alkyl-methylidene, arylmethylidene, heteroarylmethylidene, aryl-C₁₋₃-alkyl-methylidene or heteroaryl-C₁₋₃-alkyl-methylidene,
   while alkyl, alkenyl, alkynyl, cycloalkyl and cycloalkenyl groups may be partly or totally fluorinated or mono- or disubstituted by identical or different substituents selected from chlorine, cyano, hydroxy, C₁₋₃-alkoxy and CIl₃-alkyl, and
   the above-mentioned unsubstituted methylidene group or the above-mentioned monosubstituted methylidene groups may additionally be monosubstituted by fluorine, chlorine, C₁₋₃-alkyl, cyano or nitro, and
   in cycloalkyl, cycloalkenyl, cycloalkylidene and cycloalkenylidene groups one or two methylene groups may independently of one another be replaced by O, S, CO, SO, SO₂ or NR^{N}, or
   X denotes a group according to partial formula 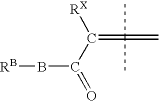
      wherein
R^{X} denotes hydrogen, fluorine, chlorine, cyano, trifluoromethyl or C₁₋₃-alkyl,
B denotes a single bond, —O— or —NR^{N}—,
R^{B} denotes hydrogen, C₁₋₆-alkyl, C₃₋₆-alkenyl, C₃₋₆-alkynyl, C₃₋₇-cycloalkyl, C₅₋₇-cycloalkenyl, C₃₋₇-cycloalkyl-C₁₋₃-alkyl, C₅₋₇-cycloalkenyl-C₁₋₃-alkyl, aryl, heteroaryl, aryl-C₁₋₃-alkyl or heteroaryl-C₁₋₃-alkyl,
   while alkyl, cycloalkyl and cycloalkenyl groups may be partly or completely fluorinated or mono- or disubstituted by identical or different substituents selected from chlorine, cyano, hydroxy, C₁₋₃-alkoxy and C₁₋₃-alkyl, or
   R^{B} and B are joined together, forming a heterocyclic ring selected from pyrrolidine, morpholine, piperidine, piperazine and 4-(C₁₋₄-alkyl)-piperazine, the heterocyclic ring being bound to the C═O— group via the imino group,
Z denotes oxygen, methylene, dimethylmethylene, difluoromethylene or carbonyl;
while the term aryl groups used in the definition of the above groups denotes phenyl or naphthyl groups, which may be mono- or disubstituted independently of one another by identical or different groups L; and
the term heteroaryl groups used in the definition of the above-mentioned groups denotes a pyrrolyl, furanyl, thienyl, pyridyl, indolyl, benzofuranyl, benzothiophenyl, quinolinyl or isoquinolinyl group,
or a pyrrolyl, furanyl, thienyl, imidazolyl or pyridyl group, wherein one or two methyne groups are replaced by nitrogen atoms,
or an indolyl, benzofuranyl, benzothiophenyl, quinolinyl or isoquinolinyl group, wherein one to three methyne groups are replaced by nitrogen atoms,
while the above-mentioned heteroaryl groups may be mono- or disubstituted independently of one another by identical or different groups L;
while by the N-heterocycloalkyl group mentioned in the definition of the above-mentioned groups is meant a saturated carbocyclic ring which comprises an imino group in the ring, which may comprise another optionally substituted imino group or an O or S atom in the ring, and
unless otherwise stated the above-mentioned alkyl groups may be straight-chain or branched,
the tautomers, the stereoisomers, the mixtures thereof and the salts thereof, particularly the physiologically acceptable salts thereof.

The compounds according to the invention of general formula I and the physiologically acceptable salts thereof have valuable pharmacological properties, particularly an inhibitory effect on the sodium-dependent glucose cotransporter SGLT, particularly SGLT2. Moreover compounds according to the invention may have an inhibitory effect on the sodium-dependent glucose cotransporter SGLT1. Compared with a possible inhibitory effect on SGLT1 the compounds according to the invention preferably inhibit SGLT2 selectively.

The present invention also relates to the physiologically acceptable salts of the compounds according to the invention with inorganic or organic acids.

Therefore, the invention also relates to the use of the compounds according to the invention, including the physiologically acceptable salts, as pharmaceutical compositions.

This invention also relates to pharmaceutical compositions, containing at least one compound according to the invention or a physiologically acceptable salt according to the invention, optionally together with one or more inert carriers and/or diluents.

A further subject of this invention is the use of at least one compound according to the invention or a physiologically acceptable salt of such a compound for preparing a pharmaceutical composition which is suitable for the treatment or prevention of diseases or conditions which can be influenced by inhibiting the sodium-dependent glucose cotransporter SGLT, particularly SGLT2.

This invention also relates to the use of at least one compound according to the invention for preparing a pharmaceutical composition which is suitable for the treatment of metabolic disorders.

This invention also relates to the use of at least one compound according to the invention for preparing a pharmaceutical composition for inhibiting the sodium-dependent glucose cotransporter SGLT, particularly SGLT2.

The invention further relates to a process for preparing a pharmaceutical composition according to the invention, characterised in that a compound according to the invention is incorporated in one or more inert carriers and/or diluents by a non-chemical method.

The present invention also relates to a process for preparing the compounds of general formula I according to the invention, characterised in that
a) in order to prepare compounds of general formula I as defined hereinbefore and hereinafter,
a compound of general formula II 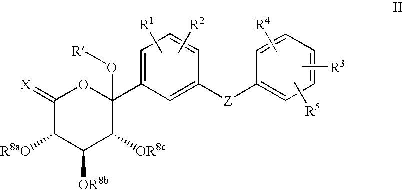
   wherein
R′ denotes H, C₄-alkyl, (C, ₁₋₁₈-alkyl)carbonyl, (C₁₋₁₈-alkyl)oxycarbonyl, arylcarbonyl or aryl-(C₁₋₃-alkyl)-carbonyl, wherein the alkyl or aryl groups may be mono- or polysubstituted by halogen;
R^{8a}, R^{8b}
R^{8c} independently of one another have one of the meanings given hereinbefore and hereinafter for the groups R^{7a}, R^{7b}, R^{7c}, denote a benzyl group or a R^{a}R^{b}R^{c}Si group or a ketal or acetal group, particularly an alkylidene or arylalkylidene ketal or acetal group, while in each case two adjacent groups R^{8a}, R ^{8b}, R^{8c}, R^{8d} may form a cyclic ketal or acetal group or a 1,2-di(C₁₋₃-alkoxy)-1,2-di(C₁₋₃-alkyl)-ethylene bridge, while the above-mentioned ethylene bridge together with two oxygen atoms and the associated two carbon atoms of the pyranose ring form a substituted dioxane ring, particularly a 2,3-dimethyl-2,3-di(C₁₋₃-alkoxy)-1,4-dioxane ring, and while alkyl, aryl and/or benzyl groups may be mono- or polysubstituted by halogen or C₁₋₃-alkoxy and benzyl groups may also be substituted by a di-(C₁₋₃-alkyl)amino group; and
R^{a}, R^{b}, R^{c} independently of one another represent C₁₋₄-alkyl, aryl or aryl-C₁₋₃-alkyl, wherein the aryl or alkyl groups may be mono- or polysubstituted by halogen;
while the term aryl groups used in the definition of the above groups denotes phenyl or naphthyl groups, preferably phenyl groups;
and wherein the groups X and R¹ to R⁵ and the bridge Z are as defined above and hereinafter;
is reacted with a reducing agent in the presence of an acid, and any protective groups present are cleaved at the same time or subsequently; or
b) in order to prepare compounds of general formula I wherein R^{7a}, R^{7b} and R^{7c} represent hydrogen,
in a compound of general formula III 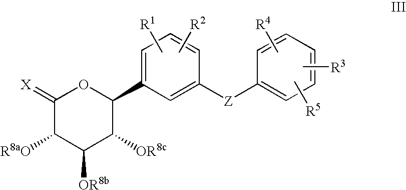
wherein X, Z, R^{8a}, R^{8b}, R^{8c} and R¹ to R⁵ are as defined above and hereinafter, and at least one of the groups R^{8a}, R^{8b} and R^{8c} does not denote hydrogen,
the groups R^{8a}, R^{8b} or R^{8c} which do not represent hydrogen are removed, particularly hydrolysed; and
if necessary any protective group used in the reactions described above according to method a) or b) is cleaved and/or
if desired a compound of general formula I thus obtained is selectively derivatised at a hydroxy group or this group is substituted and/or
if desired a compound of general formula I thus obtained is resolved into its stereoisomers and/or
if desired a compound of general formula I thus obtained is converted into the salts thereof, particularly, for pharmaceutical use, into the physiologically acceptable salts thereof.

### DETAILED DESCRIPTION OF THE INVENTION

Unless otherwise stated the groups, residues and substituents, particularly R¹ to R⁵, R^{X}, R^{B}, B, X, Z, L, R^{N}, R^{7a}, R^{7b}, R^{7c}, are defined as above and hereinafter.

If residues, substituents or groups occur several times in a compound, they may have the same or different meanings.

The group R³ is preferably in the meta or para position to the -Z- bridge, which means that compounds according to the following formulae I.1 and I.2, particularly formula I.2, are preferred:

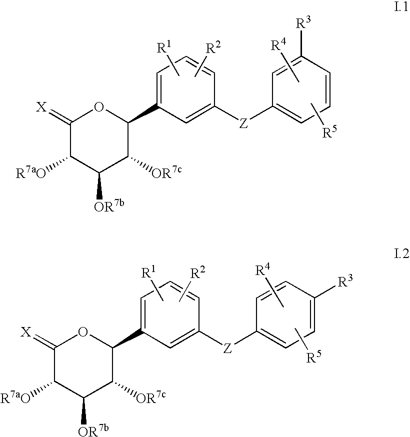

The term aryl used above and hereinafter, for example in the groups X, R^{B}, R¹ and R³, preferably denotes phenyl. According to the general definition and unless otherwise stated, the aryl group, particularly the phenyl group, may be mono- or disubstituted by identical or different groups L.

The term heteroaryl used above and hereinafter, for example in the groups X, R^{B}, R¹ and R³, preferably denotes pyridinyl, pyrimidinyl, pyridazinyl, pyrazinyl, triazinyl, imidazolyl, pyrazolyl, triazolyl, tetrazolyl, oxazolyl, oxadiazolyl, thiazolyl or thiadiazolyl. According to the general definition and unless otherwise stated, the heteroaryl group may be mono- or disubstitued by identical or different groups L.

Preferably R¹ denotes hydrogen, fluorine, chlorine, bromine, C₁₋₆-alkyl, C₂₋₆-alkynyl, C₂₋₆-alkenyl, C₃₋₇-cycloalkyl, C₅₋₇-cycloalkenyl, C₁₋₄-alkylcarbonyl, aminocarbonyl, C₁₋₄-alkylaminocarbonyl, di-(C₁₋₃-alkyl)aminocarbonyl, C₁₋₄-alkoxycarbonyl, C₁₋₄-alkylamino, di-(C₁₋₃-alkyl)amino, pyrrolidin-1-yl, piperidin-1-yl, morpholin-4-yl, C₁₋₄-alkylcarbonylamino, C₁₋₆-alkyloxy, C₃₋₇-cycloalkyloxy, C₅₋₇-cycloalkenyloxy, C₁₋₄-alkylsulphanyl, C₁₋₄-alkylsulphonyl, C₃₋₇-cycloalkylsulphanyl, C₃₋₇-cycloalkylsulphonyl, C₅₋₇-cycloalkenylsulphanyl, C₅₋₇-cycloalkenylsulphonyl, hydroxy and cyano,
while alkyl, alkenyl, alkynyl, cycloalkyl and cycloalkenyl groups may be partly or totally fluorinated or mono- or disubstituted by identical or different substituents selected from chlorine, hydroxy, C₁₋₃-alkoxy and C₁₋₃-alkyl, and
in cycloalkyl and cycloalkenyl groups one or two methylene groups may be replaced independently of one another by O, S, CO, SO or SO₂, and
in N-heterocycloalkyl groups a methylene group may be replaced by CO or SO₂.

If the group R¹ denotes a cycloalkyl or cycloalkenyl group wherein one or two methylene groups are substituted independently of one another by O, S, CO, SO or SO₂, preferred meanings of the group R¹ are selected from among tetrahydrofuranyl, tetrahydrofuranonyl, tetrahydrothienyl, tetrahydropyranyl, tetrahydropyranonyl, dioxanyl and trioxanyl.

If the group R¹ denotes an N-heterocycloalkyl group wherein a methylene group is replaced by CO or SO₂, preferred meanings of the group R¹ are selected from among pyrrolidinone, piperidinone, piperazinone and morpholinone.

Particularly preferably R¹ denotes hydrogen, fluorine, chlorine, bromine, C₁₋₆-alkyl, C₂₋₆-alkynyl, C₂₋₆-alkenyl, C₃₋₇-cycloalkyl, C₅₋₇-cycloalkenyl, C₁₋₆-alkyloxy, C₃₋₇-cycloalkyloxy or cyano, while in cycloalkyl and cycloalkenyl groups one or two methylene units may be replaced independently of one another by O or CO and alkyl, alkenyl and alkynyl groups may be partly or totally fluorinated.

Examples of the most particularly preferred groups R¹ are hydrogen, fluorine, chlorine, bromine, methyl, ethyl, isopropyl, trifluoromethyl, methoxy, cyclopentyloxy and cyano.

The group R³ preferably-denotes fluorine, chlorine, bromine, C₁₋₆-alkyl, C₂₋₆-alkynyl, C₂₋₆-alkenyl, C₃₋₇-cycloalkyl, C₃₋₇-cycloalkyl-methyl, C₅₋₇-cycloalkenyl, C₃₋₇-cycloalkenyl-methyl, aryl, heteroaryl, C₁₋₄-alkylcarbonyl, aminocarbonyl, C₁₋₄-alkylaminocarbonyl, di-(C₁₋₃-alkyl)aminocarbonyl, C₁₋₄-alkoxycarbonyl, di-(C₁₋₃-alkyl)amino, pyrrolidin-1-yl, piperidin-1-yl, morpholin-4-yl, C₁₋₄-alkylcarbonylamino, C₁₋₆-alkoxy, C₃₋₇-cycloalkyloxy, C₅₋₇-cycloalkenyloxy, aryloxy, heteroaryloxy, C₁₋₄-alkylsulphanyl, C₁₋₄-alkylsulphonyl, C₃₋₇-cycloalkylsulphanyl, C₃₋₇-cycloalkylsulphonyl, C₅₋₇-cycloalkenylsulphanyl, C₅₋₇-cycloalkenylsulphonyl, hydroxy and cyano,
while alkyl, alkenyl, alkynyl, cycloalkyl and cycloalkenyl groups may be partly or totally fluorinated or mono- or disubstituted by identical or different substituents selected from chlorine, hydroxy, C₁₋₃-alkoxy and C₁₋₃-alkyl, and
in cycloalkyl and cycloalkenyl groups one or two methylene groups may be replaced independently of one another by O, S, CO, SO or SO₂, and
in N-heterocycloalkyl groups a methylene group may be replaced by CO or SO₂,
while the terms aryl and heteroaryl are as hereinbefore defined and aryl and heteroaryl groups may be mono- or disubstituted independently of one another by identical or different groups L.

If the group R³ denotes a cycloalkyl or cycloalkenyl group wherein one or two methylene groups are replaced independently of one another by O, S, CO, SO or SO₂, preferred definitions of the group R³ are selected from among tetrahydrofuranyl, tetrahydrofuranonyl, tetrahydrothienyl, tetrahydropyranyl, tetrahydropyranonyl, dioxanyl and trioxanyl.

If the group R³ denotes an N-heterocycloalkyl group wherein a methylene group is replaced by CO or SO₂, preferred meanings of the group R³ are selected from among pyrrolidinone, piperidinone, piperazinone and morpholinone.

Particularly preferred definitions of R³ are C₁₋₆-alkyl, C₂₋₆-alkynyl, C₁₋₄-alkyloxy, C₃₋₇-cycloalkyl, C₃₋₇-cycloalkyloxy and hydroxy, while in the cycloalkyl groups one or two methylene units may be replaced independently of one another by O or CO and alkyl groups may be partly or totally fluorinated.

Most particularly preferred groups R³ are methyl, ethyl, ethynyl, isopropyl, methoxy, ethoxy, isopropyloxy, difluoromethoxy, cyclopentyloxy, tetrahydro-furan-3-yloxy and hydroxy.

A selection of the most particularly preferred examples of R³ includes methyl, ethyl, ethynyl, isopropyl, methoxy, ethoxy, difluoromethoxy, cyclopentyloxy and hydroxy.

The group X preferably denotes oxygen, methylidene, fluoromethylidene, C₁₋₆-alkyl-methylidene, C₂₋₆-alkynyl-methylidene, C₂₋₆-alkenyl-methylidene, C₃₋₇-cycloalkyl-methylidene or C₃₋₇-cycloalkylidene,
while the above-mentioned alkyl, alkenyl and alkynyl groups may be partly or completely fluorinated and independently of one another may be mono- or disubstituted by substituents selected from chlorine, hydroxy, C₁₋₃-alkoxy and C₁₋₃-alkyl, and
the above-mentioned unsubstituted methylidene group or the above-mentioned monosubstituted methylidene groups may additionally be monosubstituted by fluorine, chlorine, C₁₋₃-alkyl or cyano, and
in a cycloalkylidene group a methylene group may be replaced by O, S or NR^{N} or an ethylene group may be replaced by —NR^{N}—CO—, —CO—NR^{N}—, —O—CO— or —CO—O—,
   or
X preferably denotes a group according to partial formula T 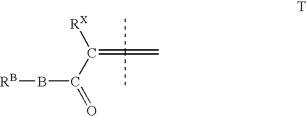
   wherein
R^{X} denotes hydrogen, fluorine, cyano, trifluoromethyl or C₁₋₃-alkyl,
B denotes a single bond, —O— or —NR^{N}—,
R^{B} denotes C₁₋₆-alkyl, C₃₋₇-cycloalkyl, C₅₋₇-cycloalkenyl, C₃₋₇-cycloalkyl-C₁₋₃-alkyl, C₅₋₇-cycloalkenyl-C₁₋₃-alkyl, aryl, heteroaryl, aryl-C₁₋₃-alkyl or heteroaryl-C₁₋₃-alkyl,
   while alkyl, cycloalkyl and cycloalkenyl groups may be partly or completely fluorinated or mono- or disubstituted by identical or different substituents selected from cyano, hydroxy, C₁₋₃-alkoxy and C₁₋₃-alkyl, or
   R^{B} and B are joined together, forming a heterocyclic ring selected from pyrrolidine, morpholine, piperidine, piperazine and 4-(C₁₋₄-alkyl)-piperazine, the heterocyclic ring being bound to the C═O— group via the imino group.

Most particularly preferred radicals of the group X are oxygen, methylidene, fluoromethylidene, difluoromethylidene, C₁₋₆-alkyl-methylidene and C₃₋₇-cycloalkylidene.

Examples of the most particularly preferred X are oxygen, methylidene, fluoromethylidene, difluoromethylidene, ethylidene, isobutylidene and cyclopentylidene.

According to a first embodiment of the compounds according to the invention X preferably denotes oxygen.

According to a second embodiment of the compounds according to the invention X preferably denotes methylidene, fluoromethylidene, C₁₋₆-alkyl-methylidene, C₂₋₆-alkynyl-methylidene, C₂₋₆-alkenyl-methylidene, C₃₋₇-cycloalkyl-methylidene or C₃₋₇-cycloalkylidene,
while the above-mentioned alkyl, alkenyl and alkynyl groups may be partly or completely fluorinated and may be mono- or disubstituted independently of one another by substituents selected from chlorine, hydroxy, C₁₋₃-alkoxy and C₁₋₃-alkyl, and
the above-mentioned unsubstituted methylidene group or the above-mentioned monosubstituted methylidene groups may additionally be monosubstituted by fluorine, chlorine, C₁₋₃-alkyl or cyano, and
in a cycloalkylidene group a methylene group may be replaced by O, S or NR^{N} or an ethylene group may be replaced by —NR^{N}—CO—, —CO—NR^{N}—, —O—CO— or —CO—O—.

In the event that in a cycloalkylidene group a methylene group is replaced by O, S or NR^{N} or an ethylene group is replaced by —NR^{N}—CO—, —CO—NR^{N}—, —O—CO— or —CO—O—, the definition of such a substituted cycloalkylidene group is preferably selected from among dihydrofuranylidene, dihydropyranylidene, dihydrothiophenylidene, pyrrolidinylidene, piperidinylidene, dihydrofuranonylidene, dihydropyranonylidene, pyrrolidinonylidene, N-methylpyrrolidinonylidene, piperidinonylidene and N-methylpiperidinonylidene.

Particularly preferred radicals of the group X according to this second embodiment are methylidene, fluoromethylidene, difluoromethylidene, C₁₋₆-alkyl-methylidene, C₃₋₇-cycloalkyl-methylidene and C₃₋₇-cycloalkylidene, particularly methylidene, fluoromethylidene, difluoromethylidene and C₁₋₄-alkyl-methylidene.

Most particularly preferred groups X are methylidene, fluoromethylidene and difluoromethylidene.

According to a third embodiment of the compounds according to the invention X preferably denotes a group according to partial formula T 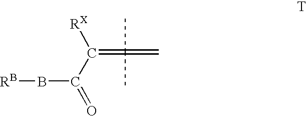
wherein
R^{X} denotes hydrogen, fluorine, cyano, trifluoromethyl or C₁₋₃-alkyl,
B denotes a single bond, —O— or —NR^{N}—,
R^{B} denotes C₁₋₆-alkyl, C₃₋₇-cycloalkyl, C₅₋₇-cycloalkenyl, C₃₋₇-cycloalkyl-C₁₋₃-alkyl, C₅₋₇-cycloalkenyl-C₁₋₃-alkyl, aryl, heteroaryl, aryl-C₁₋₃-alkyl or heteroaryl-C₁₋₃-alkyl,
   while alkyl, cycloalkyl and cycloalkenyl groups may be partly or completely fluorinated or mono- or disubstituted by identical or different substituents selected from cyano, hydroxy, C₁₋₃-alkoxy and C₁₋₃-alkyl, or
   R^{B} and B are joined together, forming a heterocyclic ring selected from pyrrolidine, morpholine, piperidine, piperazine and 4-(C₁₋₄-alkyl)-piperazine, the heterocyclic ring being bound to the C═O— group via the imino group.

Particularly preferably X has a meaning according to the above partial formula T, wherein
R^{X} denotes hydrogen or C₁₋₃-alkyl,
B denotes a single bond, —O— or —NR^{N}—,
R^{B} denotes C₁₋₆-alkyl, C₃₋₇-cycloalkyl or aryl-C₁₋₃-alkyl, or if B denotes a single bond or —NR^{N}—, R^{B} may also denote aryl,
   while alkyl and cycloalkyl groups may be partly or completely fluorinated or monosubstituted by cyano, hydroxy, C₁₋₃-alkoxy or C₁₋₃-alkyl, or
   R^{B} and B are joined together, forming a heterocyclic ring selected from pyrrolidine, morpholine, piperidine, piperazine and 4-(C₁₋₄-alkyl)-piperazine, the heterocyclic ring being bound to the C═O— group via the imino group.

Most particularly preferred definitions of the group X according to this third embodiment are selected from the partial formulae T1, T2 and T3: 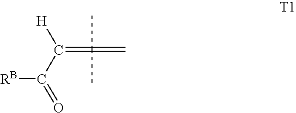
wherein R^{B} denotes methyl, ethyl, isopropyl or phenyl; 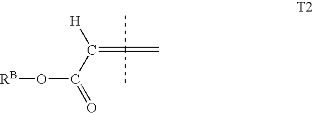
wherein R^{B} denotes methyl, ethyl, isopropyl or benzyl; or 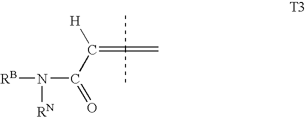
wherein R^{B} denotes methyl, ethyl, isopropyl or phenyl and RN denotes H or methyl.

If there are cycloalkyl, cycloalkenyl, cycloalkylidene or cycloalkenylidene rings wherein two methylene groups are replaced by O or S or by CO, SO or SO₂ or optionally NR^{N} in the radicals or groups X, R¹ or R³, these methylene groups are preferably not joined together directly. However, if two methylene groups are replaced by O and CO, they may be joined together directly, so that a —O—CO— or —CO—O— group is formed. If X, R¹ or R³ is a cycloalkyl, cycloalkenyl, cycloalkylidene or cycloalkenylidene group with one or two methylene groups replaced according to the invention, the relevant group X, R¹ or R³ preferably denotes a cycloalkyl, cycloalkenyl, cycloalkylidene or cycloalkenylidene group wherein a methylene group is replaced by O, S, CO, SO or SO₂ or an ethylene group is replaced by —O—CO— or —CO—O—.

Some meanings of other groups and substituents will now be given, which are to be regarded as preferred according to general formula I, formulae I.1 and I.2 and the embodiments described hereinbefore:

Preferred meanings of the group R² are hydrogen, fluorine, chlorine, bromine, methyl, hydroxy, methoxy, ethoxy, trifluoromethoxy, cyano, nitro and methyl substituted by 1 to 3 fluorine atoms.

Particularly preferred meanings of the group R² are hydrogen, fluorine, hydroxy, methoxy, ethoxy and methyl, particularly hydrogen and methyl.

If R¹ and R² are bound to two C atoms of the phenyl ring which are adjacent to one another, R¹ and R² may be joined together in such a way that R¹ and R² together preferably form a C₃₋₄ bridge, wherein one or two methylene units may be replaced independently of one another by O, NR^{N} or CO. Preferably, the groups R¹ and R² joined to one another, together with the phenyl ring by which they are joined, form a bicyclic ring system selected from among dihydroindane, dihydroindole, dihydrobenzofuran, tetrahydroquinoline, tetrahydroquinolinone, tetrahydroisoquinoline, tetrahydroisoquinolinone and tetrahydronaphthalene.

Preferred meanings of the group R⁴ are hydrogen and fluorine, particularly hydrogen.

If R³ and R⁴ are bound to two C atoms of the phenyl ring which are immediately adjacent to one another, R³ and R⁴ may be joined together in such a way that R¹ and R² together preferably form a C₃₋₄ bridge, wherein one or two methylene units may be replaced independently of one another by O, NR^{N} or CO. Preferably the interconnected groups R³ and R⁴ together with the phenyl ring by which they are joined form a bicyclic ring system selected from among dihydroindane, dihydroindole, dihydrobenzofuran, tetrahydroquinoline, tetrahydroquinolinone, tetrahydroisoquinoline, tetrahydroisoquinolinone and tetrahydronaphthalene.

Preferred meanings of the group R⁵ are hydrogen and fluorine, particularly hydrogen.

Preferred meanings of the group Z are oxygen and methylene, particularly methylene.

The substituents R^{7a}, R^{7b}, R^{7c} preferably represent, independently of one another, hydrogen, (C₁₋₈-alkyl)oxycarbonyl, (C₁₋₁₈-alkyl)carbonyl, benzoyl, particularly hydrogen or (C₁₋₆-alkyl)oxycarbonyl, (C₁₋₈-alkyl)carbonyl, particularly preferably hydrogen, methoxycarbonyl, ethoxycarbonyl, methylcarbonyl or ethylcarbonyl. Most particularly preferably R^{7a}, R^{7b} and R^{7c} denote hydrogen.

The compounds of formula I wherein R^{7a}, R^{7b} and R^{7c} have a meaning according to the invention other than hydrogen, for example C₁₋₈-alkylcarbonyl, are preferably suitable as intermediate products in the synthesis of compounds of formula I wherein R^{7a}, R^{7b} and R^{7c} denote hydrogen.

The substituents L are preferably selected independently of one another from the group consisting of fluorine, chlorine, bromine, C₁₋₃-alkyl, difluoromethyl, trifluoromethyl, C₁₋₃-alkoxy, difluoromethoxy, trifluoromethoxy and cyano, particularly preferably from the group consisting of fluorine, chlorine, methyl, trifluoromethyl, methoxy and difluoromethoxy.

Particularly preferred compounds of general formula I are selected from among the formulae I.2a to I.2d, particularly formula I.2c: 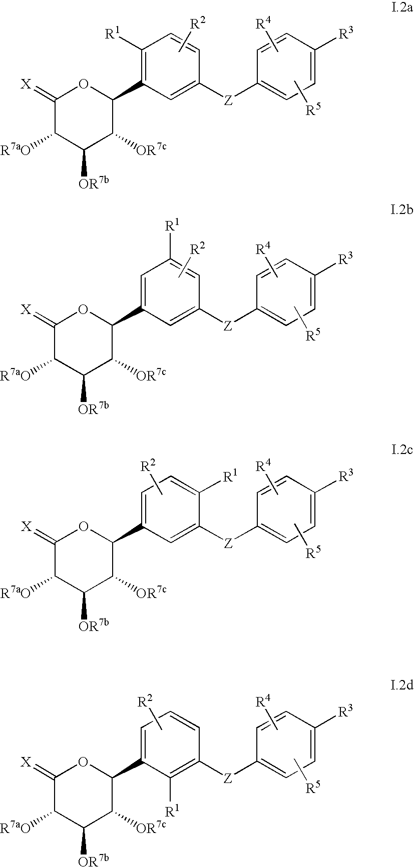
wherein R¹ to R⁵, X, Z, R^{7a}, R^{7b} R^{7c} are as hereinbefore defined.

Most particularly preferred are those compounds of formulae I.2a, I.2b, I.2c and I.2d, particularly of formula I.2c, wherein the groups R¹ to R⁵, X, Z, R^{7a}, R^{7b}, R^{7c} have the meanings stated hereinbefore as being preferred, particularly wherein
R¹ denotes hydrogen, fluorine, chlorine, bromine, C₁₋₆-alkyl, C₂₋₆-alkynyl, C₂₋₆-alkenyl, C₃₋₇-cycloalkyl, C₅₋₇-cycloalkenyl, C₁₋₆-alkyloxy, C₃₋₇-cycloalkyloxy or cyano, while in cycloalkyl and cycloalkenyl groups one or two methylene units may be replaced independently of one another by O or CO and alkyl, alkenyl and alkynyl groups may be partly or completely fluorinated, particularly preferably denotes hydrogen, fluorine, chlorine, bromine, methyl, ethyl, isopropyl, trifluoromethyl, methoxy, cyclopentyloxy or cyano, and
R³ denotes C₁₋₆-alkyl, C₂₋₆-alkynyl, C₁₋₄-alkyloxy, C₃₋₇-cycloalkyl, C₃₋₇-cycloalkyloxy or hydroxy, while in the cycloalkyl groups one or two methylene units may be replaced independently of one another by O or CO and alkyl groups may be partly or completely fluorinated, particularly preferably denotes methyl, ethyl, ethynyl, isopropyl, methoxy, ethoxy, isopropyloxy, difluoromethoxy, cyclopentyloxy, tetrahydro-furan-3-yloxy or hydroxy, and
X (1) denotes oxygen; or
   (2) denotes methylidene, fluoromethylidene, C₁₋₆-alkyl-methylidene, C₂₋₆-alkynyl-methylidene, C₂₋₆-alkenyl-methylidene, C₃₋₇-cycloalkyl-methylidene or C₃₋₇-cycloalkylidene,
      while the above-mentioned alkyl, alkenyl and alkynyl groups may be partly or completely fluorinated and may be mono- or disubstituted independently of one another by substituents selected from chlorine, hydroxy, C₁₋₃-alkoxy and C₁₋₃-alkyl,
      the above-mentioned unsubstituted methylidene group or the above-mentioned monosubstituted methylidene groups may additionally be monosubstituted by fluorine, chlorine, C₁₋₃-alkyl or cyano, and
      in a cycloalkylidene group a methylene group may be replaced by O, S or NR^{N} or an ethylene group may be replaced by —NR^{N}—CO—, —CO—NR^{N}—, —CO— or —CO—O—;
      particularly preferably X denotes methylidene, fluoromethylidene,
      difluoromethylidene, CIl₆-alkyl-methylidene or C₃₋₇-cycloalkylidene; or
   (3) denotes a group according to partial formula T 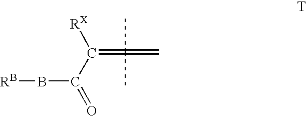
      wherein
      R^{X} denotes hydrogen, fluorine, cyano, trifluoromethyl or C₁₋₃-alkyl,
      B denotes a single bond, —O— or —NR^{N}—,
      R^{B} denotes C₁₋₆-alkyl, C₃₋₇-cycloalkyl, C₅₋₇-cycloalkenyl, C₃₋₇-cycloalkyl-C₁₋₃-alkyl, C₅₋₇-cycloalkenyl-C₁₋₃-alkyl, aryl, heteroaryl, aryl-C₁₋₃-alkyl or heteroaryl-C₁₋₃-alkyl,
      while alkyl, cycloalkyl- and cycloalkenyl groups may be partly or completely fluorinated or mono- or disubstituted by identical or different substituents selected from cyano, hydroxy, C₁₋₃-alkoxy and C₁₋₃-alkyl, or
      R^{B} and B are joined together, forming a heterocyclic ring selected from pyrrolidine, morpholine, piperidine, piperazine and 4-(C₁₋₄-alkyl)-piperazine, the heterocyclic ring being bound to the C═O— group via the imino group;
      most particularly preferably according to (3) X denotes the above-mentioned partial formula T, wherein
      R^{X} denotes hydrogen or C₁₋₃-alkyl,
      B denotes a single bond, —O— or —NR^{N}—,
      R^{B} denotes C₁₋₆-alkyl, C₃₋₇-cycloalkyl or aryl-C₁₋₃-alkyl, or in the event that B denotes a single bond or —NR^{N}—, R^{B} may also represent aryl,
      while alkyl and cycloalkyl groups may be partly or completely fluorinated or monosubstituted by cyano, hydroxy, C₁₋₃-alkoxy or C₁₋₃-alkyl, or
      R^{B} and B are joined together, forming a heterocyclic ring selected from pyrrolidine, morpholine, piperidine, piperazine and 4-(C₁₋₄-alkyl)-piperazine, the heterocyclic ring being bound to the C═O— group via the imino group; and
R² denotes hydrogen, fluorine, chlorine, bromine, methyl, hydroxy, methoxy, ethoxy, trifluoromethoxy, cyano, nitro or methyl substituted by 1 to 3 fluorine atoms, particularly preferably denotes hydrogen, fluorine, hydroxy, methoxy, ethoxy or methyl, particularly hydrogen or methyl, and
R⁴ denotes hydrogen or fluorine, particularly hydrogen, and
R⁵ denotes hydrogen or fluorine, particularly hydrogen, and
Z denotes oxygen or methylene, particularly methylene, and
R^{7a}, R^{7b},
R^{7c} independently of one another represent hydrogen, (C₁₋₈-alkyl)oxycarbonyl, (C₁₋₁₈-alkyl)carbonyl or benzoyl, particularly hydrogen or (C₁₋₆-alkyl)oxycarbonyl, (C₁₋₈-alkyl)carbonyl, particularly preferably hydrogen, methoxycarbonyl, ethoxycarbonyl, methylcarbonyl or ethylcarbonyl, most particularly preferably hydrogen, and
R^{N} independently of one another denote H or C₁₋₄-alkyl,
L independently of one another represent fluorine, chlorine, bromine, C₁₋₃-alkyl, difluoromethyl, trifluoromethyl, C₁₋₃-alkoxy, difluoromethoxy, trifluoromethoxy and cyano,
including the tautomers, stereoisomers, the mixtures thereof and the salts thereof, particularly their physiologically acceptable salts.

According to a variant of the above-mentioned embodiments, those compounds wherein the phenyl group which carries the substituent R³ comprises at least one other substituent R⁴ and/or R⁵ which is other than hydrogen are also preferred. According to this variant those compounds which comprise a substituent R⁴ representing fluorine are also preferred.

The phenyl group which carries the substituent R³ is preferably at most difluorinated.

Particularly preferred compounds of general formula I are selected from among:
(a) 1-chloro-2-(4-methoxy-benzyl)-4-(6-methylidene-β-D-xylopyranos-1-yl)-benzene
(b) 1-chloro-2-(4-methoxy-benzyl)-4-(6-fluoromethylidene-β-D-xylopyranos-1-yl)-benzene
(c) 1-chloro-2-(4-methoxy-benzyl)-4-(6-difluoromethylidene-β-D-xylopyranos-1-yl)-benzene
(d) 1-chloro-2-(4-methoxy-benzyl)-4-(6-oxo-β-D-xylopyranos-1-yl)-benzene
including the tautomers, stereoisomers and the mixtures thereof.
   Some Terms Used Above and Hereinafter to Describe the Compounds According to the Invention will Now be Defined More Closely

The term halogen denotes an atom selected from the group consisting of F, Cl, Br and I, particularly F, Cl and Br.

The term C₁₋ₙ-alkyl, wherein n may have a value of 1 to 18, denotes a saturated, branched or unbranched hydrocarbon group with 1 to n C atoms. Examples of such groups include methyl, ethyl, n-propyl, iso-propyl, butyl, iso-butyl, sec-butyl, tert-butyl, n-pentyl, iso-pentyl, neo-pentyl, tert-pentyl, n-hexyl, iso-hexyl, etc.

The term methylidene denotes a group of the partial formula 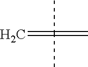
attached by a double bond.

The term C₁₋ₙ-alkyl-methylidene denotes a methylidene group wherein a hydrogen atom is substituted by a C₁₋ₙ-alkyl group.

The term C₂₋ₙ-alkynyl, wherein n has a value of 3 to 6, denotes a branched or unbranched hydrocarbon group with 2 to n C atoms and a C≡C triple bond. Examples of such groups include ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 4-methyl-2-pentynyl etc. Unless stated otherwise, alkynyl groups are linked to the rest of the molecule via the C atom in position 1. Therefore, terms such as 1-propynyl, 2-propynyl, 1-butynyl, etc. are equivalent to the terms 1-propyn-1-yl, 2-propyn-1-yl, 1-butyn-1-yl, etc. This also applies analogously to C₂₋ₙ-alkenyl groups.

The term C₁₋ₙ-alkoxy or C₁₋ₙ-alkyloxy denotes a C₁₋ₙ-alkyl-O group, wherein C₁₋ₙ-alkyl is as hereinbefore defined. Examples of such groups include methoxy, ethoxy, n-propoxy, iso-propoxy, n-butoxy, iso-butoxy, sec-butoxy, tert-butoxy, n-pentoxy, iso-pentoxy, neo-pentoxy, tert-pentoxy, n-hexoxy, iso-hexoxy etc.

The term C₁₋ₙ-alkylcarbonyl denotes a C₁₋ₙ-alkyl-C(═O) group, wherein C₁₋ₙ-alkyl is as hereinbefore defined. Examples of such groups include methylcarbonyl, ethylcarbonyl, n-propylcarbonyl, iso-propylcarbonyl, n-butylcarbonyl, iso-butylcarbonyl, sec-butylcarbonyl, tert-butylcarbonyl, n-pentylcarbonyl, iso-pentylcarbonyl, neo-pentylcarbonyl, tert-pentylcarbonyl, n-hexylcarbonyl, iso-hexylcarbonyl, etc.

The term C₃₋ₙ-cycloalkyl denotes a saturated mono-, bi-, tri- or spirocarbocyclic group with 3 to n C atoms. Examples of such groups include cyclopropyl, cyclobutyl, cyclopentyl, cyclohexyl, cycloheptyl, cyclooctyl, cyclononyl, cyclododecyl, decalin, bicyclo[3.2.1.]octyl, spiro[4.5]decyl, norpinyl, norbonyl, norcaryl, adamantyl, etc. Preferably the term C₃₋₇-cycloalkyl denotes saturated monocyclic groups.

The term C₃₋₇-cycloalkylidene denotes a C₃₋₇-cycloalkane group which is linked to the group of the molecule in question by a double bond. Examples of C₃₋₇-cycloalkylidene groups are cyclopropylidene, cyclobutylidene, cyclopentylidene, cyclohexylidene, cycloheptylidene.

The term C₃₋ₙ-cycloalkyloxy denotes a C₃₋ₙ-cycloalkyl-O group, wherein C₃₋ₙ-cycloalkyl is as hereinbefore defined. Examples of such groups include cyclopropyloxy, cyclobutyloxy, cyclopentyloxy, cyclohexyloxy, cycloheptyloxy, etc.

The term C₅₋ₙ-cycloalkenyl denotes a C₅₋ₙ-cycloalkyl group which is as hereinbefore defined and additionally comprises at least one unsaturated C═C double bond.

The term C₃₋ₙ-cycloalkylcarbonyl denotes a C₃₋ₙ-cycloalkyl-C(═O) group wherein C₃₋ₙ-cycloalkyl is as hereinbefore defined.

The term tri-(C₁₋₄-alkyl)silyl comprises silyl groups which comprises identical alkyl groups or two or three different alkyl groups.

The term di-(C₁₋₃-alkyl)amino comprises amino groups which have identical alkyl groups or two different alkyl groups.

The term N-heterocycloalkyl denotes a saturated carbocyclic ring which comprises an imino group in the ring, and which may additionally comprise another optionally substituted imino group or an O or S atom in the ring. By an imino group is meant the group —NH—. Examples of such N-heterocycloalkyl groups are pyrrolidine, piperidine, piperazine, N-alkyl-piperazine and morpholine.

If alkyl radicals occurring in groups, for example in X, R¹ or R³, may be substituted, e.g. fluorinated, this encompasses not only alkyl radicals in the groups which represent alkyl directly but also in other definitions which include alkyl groups, such as for example alkoxy, alkylcarbonyl, alkoxyalkyl, etc. Thus, for example X, R¹ and R³ representing alkoxy, wherein the alkyl groups may be partly or totally fluorinated, also include difluoromethoxy and trifluoromethoxy.

The style used above and hereinafter, in which a bond of a substituent in a phenyl group is shown towards the centre of the phenyl ring, denotes, unless otherwise stated, that this substituent may be bound to any free position of the phenyl ring bearing an H atom.

The compounds according to the invention may be obtained using methods of synthesis known in principle. Preferably the compounds are obtained by the following methods according to the invention which are described in more detail hereinafter.

The D-xylose derivatives described hereinafter may be obtained from D-glucose by replacement or suitable derivatisation of the 6-hydroxy group and subsequent substitution with the desired group. The diagrams that follow show, by way of example, some methods of synthesising the compounds claimed. The individual reactions are largely standard transformations in organic chemistry and are easily performed by the skilled man. For simplicity's sake, in the formulae that follow, the group of the partial formula 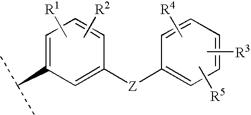
substituted according to the invention is represented by the group 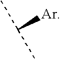
The groups PG symbolise protective groups, which may for example have one of the meanings of the groups R^{8a}, R^{8b} and R^{8c}.

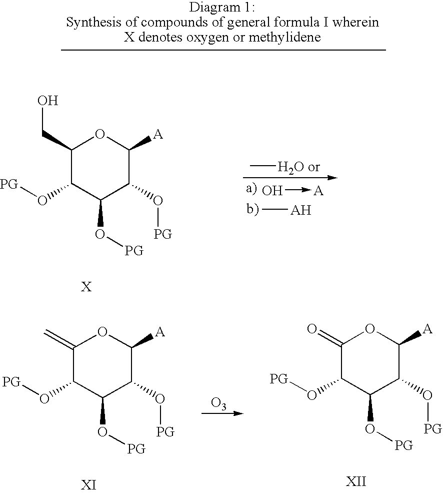

Diagram 1 shows a possible method of synthesising compounds of formula I according to the invention wherein X denotes O or methylidene. Starting from the D-glucose derivative X the 6-OH group is eliminated to form the methylidene analogue XI. This transformation may be carried out in one step with reagents such as Burgess reagent (Et₃NSO₂NCOOMe). A two-stage method of synthesis includes intermediate transformation of the OH group into a leaving group A, preferably chloride, bromide, iodide or sulphonate, such as for example tosylate, mesylate or trifluoromethylsulphonate, followed by elimination of the leaving group A with organic or inorganic bases, particularly DBU, hydroxides or alkoxides, such as for example methoxide, ethoxide or tert.butoxide (see Example 1). Moreover, the OH group may also be converted into an arylthio or arylseleno group, either directly according to a Mitsunobu variant (see Synthesis 1981, 1-28 and Org. React. 1993, 42, 335-656) or indirectly by introducing a leaving group A, and, after oxidation to form the sulphoxide (aryl-SO—) or selenium oxide (aryl-SeO—) and heating in an inert solvent such as e.g. toluene, xylene, mesitylene or dichloroethane, this arylthio or arylseleno group may be subjected to thermal syn elimination to obtain the product XI. The methylidene compound XI may be subjected to C═C— cleaving by ozonolysis, thus yielding the corresponding lactone XII. The primary or secondary ozonides resulting from the ozonolysis may be worked up either reductively, e.g. with borohydrides, dialkylsulphides or triarylphosphines, or oxidatively, e.g. with hydrogen peroxide.

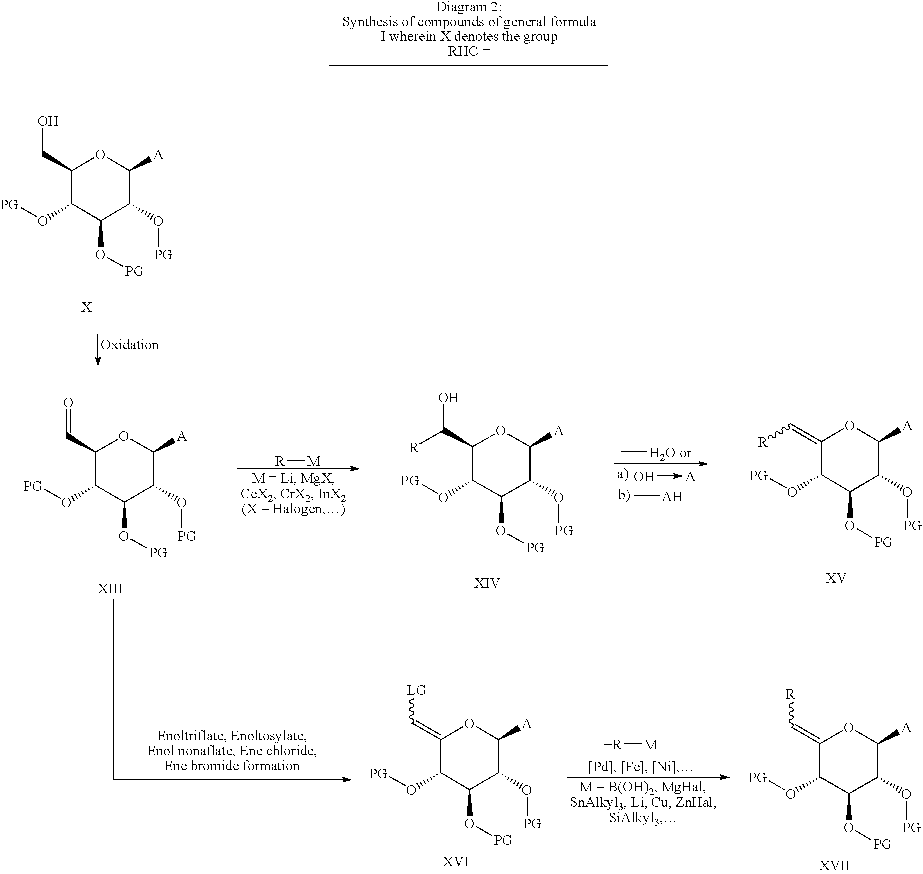

Compounds of formula I wherein the group X denotes a substituted methylidene group ═CHR, where R denotes C₁₋₆-alkyl, C₂₋₆-alkenyl, C₂₋₆-alkynyl, C₃₋₇-cycloalkyl, C₅₋₇-cycloalkenyl, aryl, heteroaryl, aryl-C₁₋₃-alkyl or heteroaryl-C₁₋₃-alkyl, in which the alkyl, alkenyl, alkynyl, cycloalkyl and cycloalkenyl groups may be partly or completely fluorinated or mono- or disubstituted by identical or different substituents selected from chlorine, cyano, hydroxy, C₁₋₃-alkoxy and C₁₋₃-alkyl, and in which in the cycloalkyl and cycloalkenyl groups one or two methylene groups may be replaced independently of one another by O, S, CO, SO, SO₂ or NR^{N}, may be prepared according to Diagram 2. According to Diagram 2 the D-glucose derivative X is oxidised at the 6-OH group to form the aldehyde XIII. Suitable oxidising agents are in particular DMSO and oxalyl chloride, as described for example according to Swern, Dess-Martin periodinane, manganese dioxide, chromium(VI) reagents such as for example PCC or potassium dichromate, tetramethylpiperidine oxide (TEMPO) or perrhutenate. The group R may be inserted by the addition of a corresponding organometallic compound such as for example a lithium, magnesium, cerium, zinc, chromium or indium compound, which may carry one or more of the corresponding group R, to yield the alcohol XIV (see M. Schlosser, Organometallics in Synthesis, John Wiley & Sons, Chichester/N.Y. Brisbane/Toronto/Singapore, 1994). The target compound XV may be obtained therefrom by dehydration using the methods already described for the transformation of X into XI. Depending on whether the group R is added stereoselectively, only one double bond isomer or a mixture of the two is obtained.

Using a second method of synthesis, the olefin XVI, which carries a transition metal-activatable group LG such as for example a tosylate, triflate, nonaflate, chlorine, bromine or iodine, is generated from the aldehyde XIII, if necessary using a base such as triethylamine. Transition metals such as e.g. palladium, nickel, rhodium, copper or iron enable the activated function LG in XVI to be replaced with groups R, which may be introduced into the coupling reaction after being bound, for example, to silicon (e.g. Hiyama coupling), boron (e.g. Suzuki coupling), tin (e.g. Stille coupling), zinc or zirconium (e.g. Negishi coupling), magnesium (e.g. Kumada coupling or Kochi coupling), lithium (e.g. Kumada coupling), aluminium, indium, chromium (e.g. Nozaki-Hiyama-Kishi reaction) or copper (e.g. Sonogashira coupling) (see L. Brandsma, S. F. Vasilevsky, H. D. Verkruijsse, Application of Transition Metal Catalysts in Organic Synthesis, Springer-Verlag, Berlin/Heidelberg, 1998). If R carries a double bond, XVI may also be attached by a Heck reaction or a variant thereof using a nickel, palladium or rhodium catalyst. The group R is introduced stereoselectively with retention and yields the product XVII with the E or Z configuration as a function of the configuration of the double bond in the adduct XVI (see Chem. Rev. 2000, 100 (8), 3009-3066).

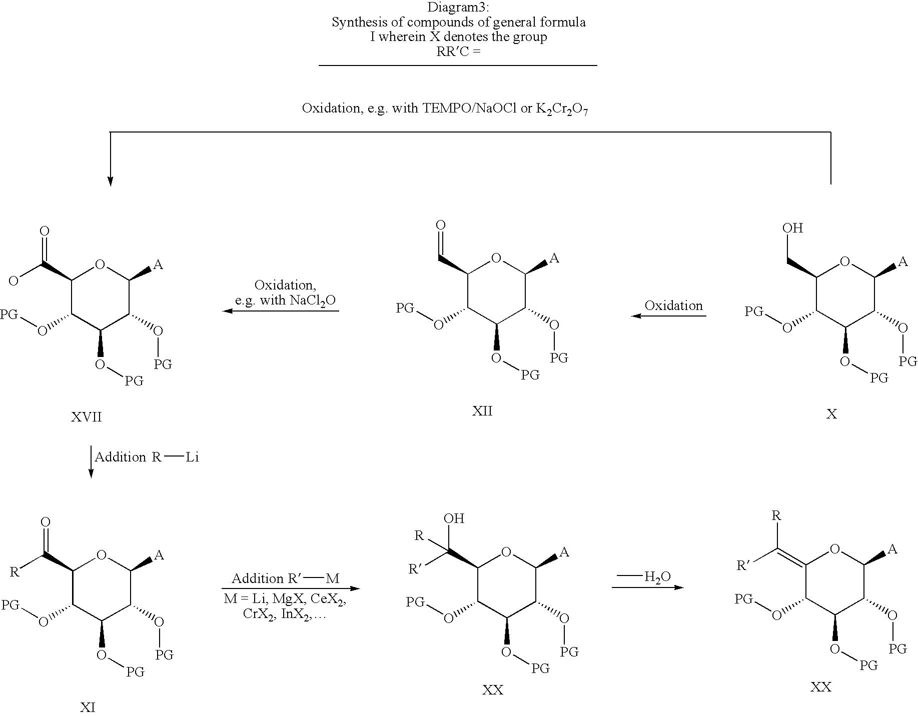

Diagram 3 illustrates the preparation of disubstituted methylidenepyran derivatives, wherein R is as hereinbefore defined and R′ is C₁₋₃-alkyl or cyano. R′ and R may also be joined together and together form a C₃₋₇-cycloalkylidene or C₅₋₇-cycloalkenylidene unit, while in the cycloalkylidene and cycloalkenylidene groups one or two methylene groups may be replaced independently of one another by O, S, CO, SO, SO₂ or NR^{N}. Starting from the aldehyde XIII (which may be prepared analogously to Diagram 2) the carboxylic acid XVIII is prepared by oxidation with e.g. sodium chlorite. The carboxylic acid may also be obtained from the glucose derivative X by oxidation with tetramethylpiperidine oxide (TEMPO) and hypochloride or with potassium dichromate. The carboxylic acid (—CO—OH— group) may be converted into the corresponding grapevine amide (—CO-NMe-OMe—group), to which the group R may be mono-added as an R-metal compound, the metal being e.g. lithium or magnesium, to produce the ketone XIX. Alternatively the carboxylic acid chloride may also be prepared from the carboxylic acid, to which again an R-metal compound may be added analogously, and this may be done in the presence of a catalyst such as palladium, copper, nickel or iron. However, the carboxylic acid XVIII may itself be directly reacted further with organolithium compounds which carry R, to form the ketone XIX. In another step, a second group R¹ may be attached to the ketone XIX by the addition of a corresponding organometallic compound to yield the alcohol XX. The organometallic compound may in this case be a lithium, Grignard, chromium or cerium compound, for example. The second organometal reaction may also be an intramolecular addition from the group R onto the ketone, producing the corresponding cycloalkanols or cycloalkenols. The last reaction step of the sequence is again a dehydration, which may be carried out as described above.

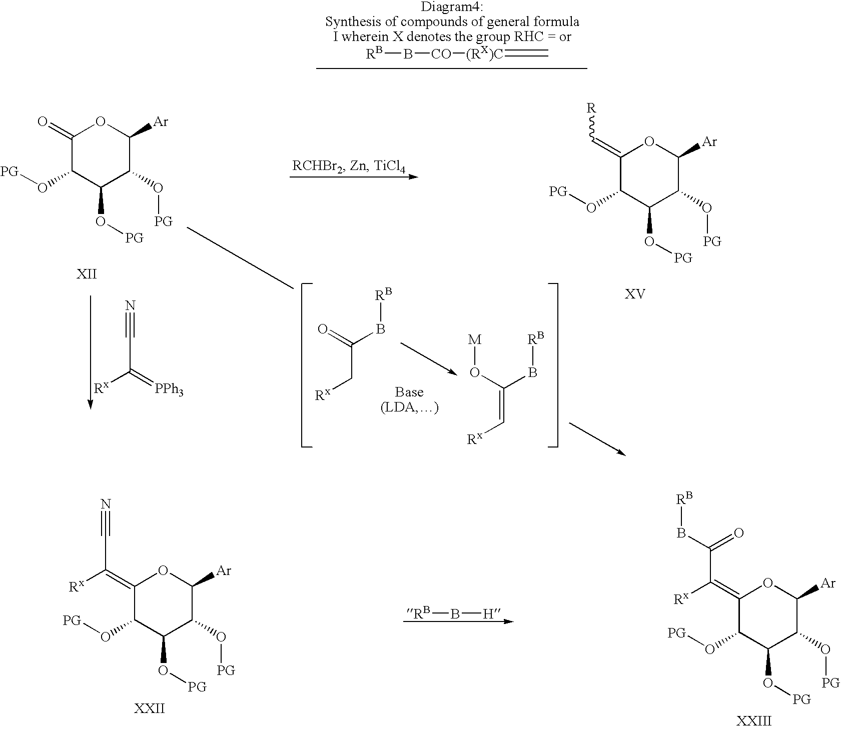

Diagram 4 shows another alternative method of preparing compounds of type XV which starts from the lactone XII, obtainable analogously to Diagram 1. The lactone XII is reacted with a mixture of the desired group R, which is in the form of a dihalide, e.g. dibromide or diiodide, zinc and titanium tetrachloride (see J. Org. Chem. 1987, 52 (19), 4410-4412). In the same way, the fluoro- and difluoromethylidene compound may be prepared by reacting the lactone XII with FCHBr₂ or F₂CBr₂, (Me₂N)₃P and Zn (see J. Chem. Soc., Chem. Commun. 1989, 19, 1437-1439).

The bottom half of Diagram 4 shows the preparation of the compounds according to the invention with the group X representing R^{B}—B—COCR^{X}═. Here, R^{B} and B are as hereinbefore defined and R^{X} is hydrogen, fluorine, chlorine, C₁₋₃-alkyl, cyano and nitro. Direct access to the target compounds XXIII starts from the lactone XII, which is reacted with the enolate from R^{B}—B—COCH₂R^{X}, which may be obtained for example by reaction with lithium diisopropylamide (LDA) or lithium hexamethyldisilazide. An alternative method of synthesising XXIII proceeds e.g. via the nitrile XXII, which may be obtained from the lactone XII by reacting with triphenyl phosphanylidene acetonitrile or a derivative thereof (see Tetrahedron Asymmetry 2000, 11 (2), 417-421). Addition of the corresponding R^{B}—B groups as anions or neutral compounds such as e.g. R^{B}—O⁻/R^{B}—OH, R^{B}—NR^{N-}—/R^{B}—NR^{N}H or R^{B-}/R^{B}—H then yield the target compounds XXIII, after hydrolysis of the imine intermediate. Hydrolysis of the nitrile compound XXII to form the corresponding carboxylic acid and additions to the latter or activated derivatives thereof, such as e.g. carboxylic acid chloride or anhydride, may also lead to the product XXIII.

In another possible method of preparing compounds of general formula I, a compound of general formula II 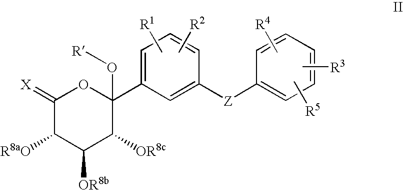
wherein X, Z and R′, R¹ to R⁵ are as hereinbefore defined and
R^{8a}, R^{8b} and R^{8c} are as hereinbefore defined and independently of one another represent acetyl, pivaloyl, benzoyl, tert-butoxycarbonyl, benzyloxycarbonyl, trialkylsilyl, benzyl or substituted benzyl, for example,
is reacted with a reducing agent in the presence of an acid.

Suitable reducing agents for the reaction include for example silanes, such as triethyl, tripropyl, triisopropyl or diphenyl silane, sodium borohydride, sodium cyanoborohydride, zinc borohydride, borane, lithium aluminium hydride, diisobutylaluminium hydride or samarium iodide. The reductions are preferably carried out in the presence of a suitable acid, such as e.g. hydrochloric acid, toluenesulphonic acid, trifluoroacetic acid, acetic acid, boron trifluoride etherate, trimethylsilyl triflate, titanium tetrachloride, tin tetrachloride, scandium triflate or zinc iodide. Depending on the reducing agent and the acid the reaction may be carried out in a solvent, such as for example methylene chloride, chloroform, acetonitrile, toluene, hexane, diethyl ether, tetrahydroftiran, dioxane, ethanol, water or mixtures thereof at temperatures between −60° C. and 120° C. A particularly suitable combination of reagents consists for example of triethylsilane and boron trifluoride etherate, which is expediently used in acetonitrile or dichloromethane at temperatures from −60° C. to 60° C. In addition, hydrogen may be used for the transformation described, in the presence of a transition metal catalyst such as e.g. palladium on charcoal or Raney nickel, in solvents such as tetrahydrofuran, ethyl acetate, methanol, ethanol, water or acetic acid.

Alternatively, in order to prepare compounds of general formula I according to method b) of the invention, in a compound of general formula III 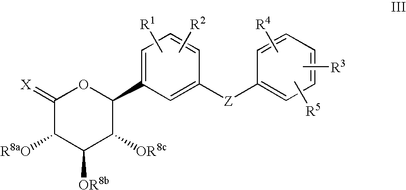
wherein X, Z and R¹ to R⁵ are as hereinbefore defined and
R^{8a} to R^{8c} represent one of the protective groups defined hereinbefore, such as e.g. an acyl, arylmethyl, acetal, ketal or silyl group, the protective groups are cleaved.

Any acyl, acetal or ketal protecting group used is cleaved hydrolytically, for example, in an aqueous solvent, e.g. in water, isopropanol/water, acetic acid/water, tetrahydrofuran/water or dioxane/water, in the presence of an acid such as trifluoroacetic acid, hydrochloric acid or sulphuric acid or aprotically, e.g. in the presence of iodotrimethylsilane, at temperatures between 0 and 120° C., preferably at temperatures between 10 and 100° C. An acyl group may also be cleaved in the presence of an alkali metal base such as lithium hydroxide, sodium hydroxide or potassium hydroxide. A trifluoroacetyl group is preferably cleaved by treatment with an acid such as hydrochloric acid, optionally in the presence of a solvent such as acetic acid, at temperatures between 50 and 120° C. or by treatment with sodium hydroxide solution, optionally in the presence of a solvent such as tetrahydrofuran or methanol, at temperatures between 0 and 50° C.

A trimethylsilyl group is cleaved for example in water, an aqueous solvent mixture or a lower alcohol such as methanol or ethanol in the presence of a base such as lithium hydroxide, sodium hydroxide, potassium carbonate or sodium methoxide. In aqueous or alcoholic solvents, acids, such as e.g. hydrochloric acid, trifluoroacetic acid or acetic acid are also suitable, for example. Fluoride reagents, such as e.g. tetrabutylammonium fluoride, are also suitable for cleaving in organic solvents, such as for example diethyl ether, tetrahydrofuran or dichloromethane.

A benzyl, methoxybenzyl or benzyloxycarbonyl group is advantageously cleaved hydrogenolytically, e.g. with hydrogen in the presence of a catalyst such as palladium/charcoal, in a suitable solvent such as methanol, ethanol, ethyl acetate or glacial acetic acid, optionally with the addition of an acid such as hydrochloric acid, at temperatures between 0 and 100° C., but preferably at ambient temperature between 20 and 60° C., and under a hydrogen pressure of 1 to 7 bar, but preferably from 3 to 5 bar. However, a 2,4-dimethoxybenzyl group is preferably cleaved in trifluoroacetic acid in the presence of anisole.

A tert.-butyl or tert.-butyloxycarbonyl group is preferably cleaved by treatment with an acid such as trifluoroacetic acid or hydrochloric acid or by treatment with iodotrimethylsilane, optionally using a solvent such as methylene chloride, dioxane, methanol or diethyl ether.

In the reactions described hereinbefore, any reactive groups present such as ethynyl, hydroxy, amino, alkylamino or imino groups may be protected during the reaction by conventional protecting groups which are cleaved again after the reaction, e.g. as described above.

For example, a protecting group for an ethynyl group may be a trimethylsilyl or triisopropyl group. The 2-hydroxyisoprop-2-yl group may also be used as a protective group.

For example, a protecting group for a hydroxy group may be a trimethylsilyl, acetyl, trityl, benzyl or tetrahydropyranyl group.

Examples of protecting groups for an amino, alkylamino or imino group include the formyl, acetyl, trifluoroacetyl, ethoxycarbonyl, tert.-butoxycarbonyl, benzyloxycarbonyl, benzyl, methoxybenzyl or 2,4-dimethoxybenzyl group.

Furthermore, the compounds of general formula I thus obtained may be selectively derivatised at a hydroxy group or the hydroxy group itself may be substituted (see Examples VII, VIII, 1, 2, 4, 5, 6).

Moreover, the compounds of general formula I obtained may be resolved into their enantiomers and/or diastereomers, as mentioned hereinbefore. Thus, for example, cis/trans mixtures may be resolved into their cis and trans isomers, and compounds with at least one optically active carbon atom may be separated into their enantiomers.

Thus, for example, the cis/trans mixtures may be resolved by chromatography into the cis and trans isomers thereof, the compounds of general formula I obtained which occur as racemates may be separated by methods known per se (cf. Allinger N. L. and Eliel E. L. in “Topics in Stereochemistry”, Vol. 6, Wiley Interscience, 1971) into their optical antipodes and compounds of general formula I with at least 2 asymmetric carbon atoms may be resolved into their diastereomers on the basis of their physical-chemical differences using methods known per se, e.g. by chromatography and/or fractional crystallisation, and, if these compounds are obtained in racemic form, they may subsequently be resolved into the enantiomers as mentioned above.

The enantiomers are preferably separated by column separation on chiral phases or by recrystallisation from an optically active solvent or by reacting with an optically active substance which forms salts or derivatives such as e.g. esters or amides with the racemic compound, particularly acids and the activated derivatives or alcohols thereof, and separating the diastereomeric mixture of salts or derivatives thus obtained, e.g. on the basis of their differences in solubility, whilst the free antipodes may be released from the pure diastereomeric salts or derivatives by the action of suitable agents. Optically active acids in common use are e.g. the D- and L-forms of tartaric acid or dibenzoyltartaric acid, di-o-tolyltartaric acid, malic acid, mandelic acid, camphorsulphonic acid, glutamic acid, aspartic acid or quinic acid. An optically active alcohol may be for example (+) or (−)-menthol and an optically active acyl group in amides, for example, may be a (+)—or (−)-menthyloxycarbonyl.

Furthermore, the compounds of formula I obtained may be converted into the salts thereof, particularly for pharmaceutical use into the physiologically acceptable salts with inorganic or organic acids. Acids which may be used for this purpose include for example hydrochloric acid, hydrobromic acid, sulphuric acid, methanesulphonic acid, phosphoric acid, fumaric acid, succinic acid, lactic acid, citric acid, tartaric acid or maleic acid.

Moreover, the compounds obtained may be converted into mixtures, for example 1:1 or 1:2 mixtures with amino acids, particularly with alpha-amino acids such as proline or phenylalanine, which may have particularly favourable properties such as a high crystallinity.

The compounds of general formulae II and III used as starting materials are partly known from the literature or may be obtained by methods known from the literature and also analogously to the methods described in the Examples, optionally with the additional inclusion of protecting groups.

The compounds according to the invention may advantageously also be obtained by the methods described in the following Examples, which may also be combined with methods known to the skilled man from the literature, for example, particularly the methods described in WO 98/31697, WO 01/27128, WO 02/083066, WO 03/099836, WO 04/063209 and WO 04/76470.

As already mentioned hereinbefore, the compounds of general formula I according to the invention and the physiologically acceptable salts thereof have valuable pharmacological properties, particularly an inhibitory effect on the sodium-dependent glucose cotransporter SGLT, preferably SGLT2.

The biological properties of the new compounds may be investigated as follows:

The ability of the substances to inhibit the SGLT-2 activity may be demonstrated in a test set-up in which a CHO—K1 cell line (ATCC No. CCL 61) or alternatively an HEK293 cell line (ATCC No. CRL-1573), which is stably transfected with an expression vector pZeoSV (Invitrogen, EMBL accession number L36849), which contains the cDNA for the coding sequence of the human sodium glucose cotransporter 2 (Genbank Acc. No. NM_{—}003041) (CHO-hSGLT2 or HEK-hSGLT2). These cell lines transport ¹⁴C-labelled alpha-methyl-glucopyranoside (¹⁴C-AMG, Amersham) into the interior of the cell in sodium-dependent manner.

The SGLT-2 assay is carried out as follows:
CHO-hSGLT2 cells are cultivated in Ham's F12 Medium (BioWhittaker) with 10% foetal calf serum and 250 μg/ml zeocin (Invitrogen), and HEK293-hSGLT2 cells are cultivated in DMEM medium with 10% foetal calf serum and 250 μg/ml zeocin (Invitrogen).

The cells are detached from the culture flasks by washing twice with PBS and subsequently treating with trypsin/EDTA. After the addition of cell culture medium the cells are centrifuged, resuspended in culture medium and counted in a Casy cell counter. Then 40,000 cells per well are seeded into a white, 96-well plate coated with poly-D-lysine and incubated overnight at 37° C., 5% CO₂. The cells are washed twice with 250 μl of assay buffer (Hanks Balanced Salt Solution, 137 mM NaCl, 5.4 mM KCl, 2.8 mM CaCl₂, 1.2 mM MgSO₄ and 10 mM HEPES (pH7.4), 50 μg/ml of gentamycin). 300 μl of assay buffer and 5 μl of test compound are then added to each well and the plate is incubated for a further 15 minutes in the incubator. 5 μl of 10% DMSO are used as the negative control. The reaction is started by adding 5 μl of ¹⁴C-AMG (0.05 μCi) to each well. After 2 hours' incubation at 37° C., 5% CO₂, the cells are washed again with 300 μl of PBS (20° C.) and then lysed by the addition of 25 μl of 0.1 N NaOH (5 min. at 37° C.). 200 μl of MicroScint20 (Packard) are added to each well and incubation is continued for a further 20 min at 37° C. After this incubation the radioactivity of the ¹⁴C-AMG absorbed is measured in a Topcount (Packard) using a ¹⁴C scintillation program.

To determine the selectivity with respect to human SGLT1 an analogous test is set up in which the cDNA for hSGLT1 (Genbank Acc. No. NM000343) instead of hSGLT2 cDNA is expressed in CHO—K1 or HEK293 cells.

Alternatively, measurement of the cellular membrane potential for hSGLT1 and hSGLT2 may also be used for the biological testing of substances. The cell models described earlier may be used for this. For the test, 10,000 cells per well of a black 384-well plate with a transparent base coated with poly-D-lysine are seeded in culture medium and incubated for 16 hours at 37° C., 5% CO₂. Then the cells are washed twice with glucose-free HBSS buffer (12.67 mol/l CaCl₂, 4.93 mmol/l MgCl₂, 4.07 mmol/l MgSO₄, 4.41 mmol/l KH₂PO₄; pH 7.4) and covered with 20 μl HBSS. After the addition of 20 μl of charging buffer (Membrane Potential Assay Kit Explorer R8126, Molecular Devices GmbH, Ismaning) and 20 μl of the substance to be tested in a suitable concentration, incubation is continued for a further 30 min. at 37° C., 5% CO₂. The measurement is carried out in the Fluorescent Imaging Plate Reader (Molecular Devices GmbH, Ismaning) at an excitation wavelength of 485 nm and is started by the addition of 20 μl of stimulant buffer (140 mM NaCl and 120 mM glucose). The depolarisation of the cell caused by the glucose-induced influx of Na⁺ can be measured and quantified as a change in fluorescence.

The compounds of general formula I according to the invention may for example have EC50 values of less than 1000 nM, particularly less than 200 nM, particularly preferably less than 50 nM.

In view of their ability to inhibit the SGLT activity, the compounds of general formula I according to the invention and the corresponding pharmaceutically acceptable salts thereof are theoretically suitable for the treatment and/or preventative treatment of all those conditions or diseases which may be affected by the inhibition of the SGLT activity, particularly the SGLT-2 activity. Therefore, compounds according to the invention are particularly suitable for the prevention or treatment of diseases, particularly metabolic disorders, or conditions such as type 1 and/or type 2 diabetes mellitus, complications of diabetes (such as e.g. retinopathy, nephropathy or neuropathies, diabetic foot, ulcers, macroangiopathies), metabolic acidosis or ketosis, reactive hypoglycaemia, hyperinsulinaemia, glucose metabolic disorder, insulin resistance, metabolic syndrome, dyslipidaemias of different origins, atherosclerosis and related diseases, obesity, high blood pressure, chronic heart failure, oedema and hyperuricaemia. These substances are also suitable for preventing beta-cell degeneration such as e.g. apoptosis or necrosis of pancreatic beta cells. The substances are also suitable for improving or restoring the functionality of pancreatic cells, and also for increasing the number and size of pancreatic beta cells. The compounds according to the invention may also be used as diuretics or antihypertensives and are suitable for the prevention and treatment of acute renal failure.

In particular, the compounds according to the invention, including the physiologically acceptable salts thereof, are suitable for the prevention or treatment of diabetes, particularly type 1 and type 2 diabetes mellitus, and/or diabetic complications.

The dosage required to achieve the corresponding activity for treatment or prevention usually depends on the compound which is to be administered, the patient, the nature and gravity of the illness or condition and the method and frequency of administration and is for the patient's doctor to decide. Expediently, the dosage may be from 1 to 100 mg, preferably 1 to 30 mg, by intravenous route, and 1 to 1000 mg, preferably 1 to 100 mg, by oral route, in each case administered 1 to 4 times a day. For this purpose, the compounds of formula I prepared according to the invention may be formulated, optionally together with other active substances, together with one or more inert conventional carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof, to produce conventional galenic preparations such as plain or coated tablets, capsules, powders, solutions, suspensions or suppositories.

The compounds according to the invention may also be used in conjunction with other active substances, particularly for the treatment and/or prevention of the diseases and conditions mentioned above. Other active substances which are suitable for such combinations include, in particular, those which potentiate the therapeutic effect of an SGLT inhibitor according to the invention with respect to one of the indications mentioned and/or which allow the dosage of an SGLT inhibitor according to the invention to be reduced. Therapeutic agents which are suitable for such a combination include, for example, antidiabetic agents such as metformin, sulphonylureas (e.g. glibenclamide, tolbutamide, glimepiride), nateglinide, repaglinide, thiazolidinediones (e.g. rosiglitazone, pioglitazone), PPAR-gamma-agonists (e.g. GI 262570) and antagonists, PPAR-gamma/alpha modulators (e.g. KRP 297), alpha-glucosidase inhibitors (e.g. acarbose, voglibose), DPPIV inhibitors (e.g. LAF237, MK-431), alpha2-antagonists, insulin and insulin analogues, GLP-1 and GLP-1 analogues (e.g. exendin-4) or amylin. Other active substances which are suitable as combination partners include inhibitors of protein tyrosinephosphatase 1, substances that affect deregulated glucose production in the liver, such as e.g. inhibitors of glucose-6-phosphatase, or fructose-1,6-bisphosphatase, glycogen phosphorylase, glucagon receptor antagonists and inhibitors of phosphoenol pyruvate carboxykinase, glycogen synthase kinase or pyruvate dehydrokinase, lipid lowering agents such as for example HMG-CoA-reductase inhibitors (e.g. simvastatin, atorvastatin), fibrates (e.g. bezafibrate, fenofibrate), nicotinic acid and the derivatives thereof, PPAR-alpha agonists, PPAR-delta agonists, ACAT inhibitors (e.g. avasimibe) or cholesterol absorption inhibitors such as, for example, ezetimibe, bile acid-binding substances such as, for example, cholestyramine, inhibitors of ileac bile acid transport, HDL-increasing compounds such as CETP inhibitors or ABC1 regulators or active substances for treating obesity, such as sibutramine or tetrahydrolipostatin, dexfenfluramine, axokine, antagonists of the cannabinoid 1 receptor, MCH-1 receptor antagonists, MC4 receptor agonists, NPY5 or NPY2 antagonists or β33-agonists such as SB-418790 or AD-9677 and agonists of the 5HT2c receptor.

Moreover, combinations with drugs for influencing high blood pressure, chronic heart failure or atherosclerosis such as e.g. A-II antagonists or ACE inhibitors, ECE inhibitors, diuretics, 3-blockers, Ca-antagonists, centrally acting antihypertensives, antagonists of the alpha-2-adrenergic receptor, inhibitors of neutral endopeptidase, thrombocyte aggregation inhibitors and others or combinations thereof are suitable. Examples of angiotensin II receptor antagonists are candesartan cilexetil, potassium losartan, eprosartan mesylate, valsartan, telmisartan, irbesartan, EXP-3174, L-158809, EXP-3312, olmesartan, medoxomil, tasosartan, KT-3-671, GA-0113, RU-64276, EMD-90423, BR-9701, etc. Angiotensin II receptor antagonists are preferably used for the treatment or prevention of high blood pressure and complications of diabetes, often combined with a diuretic such as hydrochlorothiazide.

A combination with uric acid synthesis inhibitors or uricosurics is suitable for the treatment or prevention of gout.

A combination with GABA-receptor antagonists, Na-channel blockers, topiramat, protein-kinase C inhibitors, advanced glycation end product inhibitors or aldose reductase inhibitors may be used for the treatment or prevention of complications of diabetes.

The dosage for the combination partners mentioned above is usefully ⅕ of the lowest dose normally recommended up to 1/1 of the normally recommended dose.

Therefore, in another aspect, this invention relates to the use of a compound according to the invention or a physiologically acceptable salt of such a compound combined with at least one of the active substances described above as a combination partner, for preparing a pharmaceutical composition which is suitable for the treatment or prevention of diseases or conditions which can be affected by inhibiting the sodium-dependent glucose cotransporter SGLT. These are preferably metabolic diseases, particularly one of the diseases or conditions listed above, most particularly diabetes or diabetic complications.

The use of the compound according to the invention, or a physiologically acceptable salt thereof, in combination with another active substance may take place simultaneously or at staggered times, but particularly within a short space of time. If they are administered simultaneously, the two active substances are given to the patient together; while if they are used at staggered times the two active substances are given to the patient within a period of less than or equal to 12 hours, but particularly less than or equal to 6 hours.

Consequently, in another aspect, this invention relates to a pharmaceutical composition which comprises a compound according to the invention or a physiologically acceptable salt of such a compound and at least one of the active substances described above as combination partners, optionally together with one or more inert carriers and/or diluents.

Thus, for example, a pharmaceutical composition according to the invention comprises a combination of a compound of formula I according to the invention or a physiologically acceptable salt of such a compound and at least one angiotensin II receptor antagonist optionally together with one or more inert carriers and/or diluents.

The compound according to the invention, or a physiologically acceptable salt thereof, and the additional active substance to be combined therewith may both be present together in one formulation, for example a tablet or capsule, or separately in two identical or different formulations, for example as a so-called kit-of-parts.

In the foregoing and following text, H atoms of hydroxyl groups are not explicitly shown in every case in structural formulae. The Examples that follow are intended to illustrate the present invention without restricting it:

Preparation of the starting compounds:

### EXAMPLE I

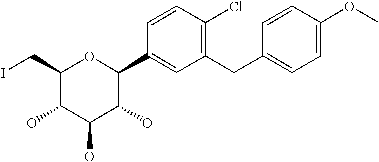

### 1-chloro-2-(4-methoxy-benzyl)-4-(6-desoxy-6-iodo-β-D-glucopyranos-1-yl)-benzene

0.53 g of triphenylphosphine, 0.13 g of imidazole and 0.48 g of iodine are added to a solution of 0.60 g of 1-chloro-2-(4-methoxy-benzyl)-4-(1-β-D-glucopyranosyl)-benzene in 5 ml of dichloromethane. The solution is stirred for 18 h at ambient temperature and then diluted with 30 ml dichloromethane. The solution is washed with 1 M hydrochloric acid, dried over sodium sulphate and freed from the solvent. The residue is purified on silica gel (dichloromethane/methanol 1:0->20:1).

Yield: 0.66 g (87% of theory)

Mass spectrum (ESI⁺): m/z=522/524 (chlorine) [M+NH₄]⁺

Preparation of the final compounds:

### EXAMPLE 1

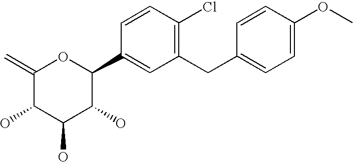

### 1-chloro-2-(4-methoxy-benzyl)-4-(6-methylidene-β-D-xylopyranos-1-yl)-benzene

0.15 g of sodium methoxide are added to an ice-cooled solution of 0.15 g of 1-chloro-2-(4-methoxy-benzyl)-4-(6-desoxy-6-iodo-β-D-glucopyranos-1-yl)-benzene in 2.5 ml of methanol. The solution is stirred for 2 h at ambient temperature and then for 16 h at 45° C. The solution is then neutralised with 1 M hydrochloric acid, extracted with dichloromethane and the organic phase is dried over sodium sulphate. The solvent is then removed and the residue is chromatographed on silica gel (dichloromethane/methanol 1:0−>15:1).

Yield: 0.062 g (83% of theory)

Mass spectrum (ESI⁺): m/z=394/396 (chlorine) [M+NF₄₁]⁺

### EXAMPLE 2

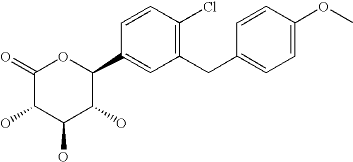

### (3S,4R,5R,6S)-6-[4-chloro-3-(4-methoxy-benzyl)-phenyl]-3,4,5-trihydroxy-tetrahydro-pyran-2-one

A solution of 0.33 g 1-chloro-2-(4-methoxy-benzyl)-4-(6-methylidene-β-D-xylopyranos-1-yl)-benzene in 20 ml dichloromethane and 5 ml of tetrahydrofuran is cooled to −78° C. Then ozone is piped through the solution until the solution has taken on a blue coloration. Oxygen is then piped through the solution until the solution has become colourless again. Then 0.32 g of triphenylphosphine are added, and the solution is stirred for 1 h at ambient temperature. After the solvent has been eliminated the residue is chromatographed on silica gel (dichloromethane/methanol 1:0->10:1).

Yield: 0,10 g (30% of theory)

Mass spectrum (ESI⁻): m/z=377/379 (chlorine) [M−H]⁻

The following compounds are also prepared analogously to the foregoing Examples and other methods known from the literature:

| No. | Structure |
|---|---|
| (2) | 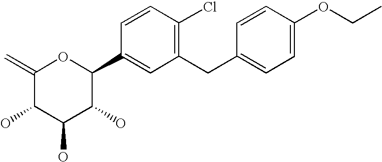 |
| (3) | 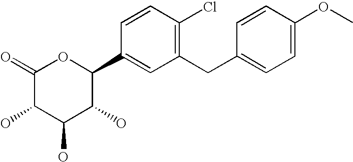 |
| (4) | 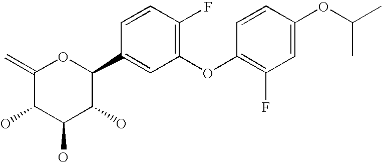 |
| (5) | 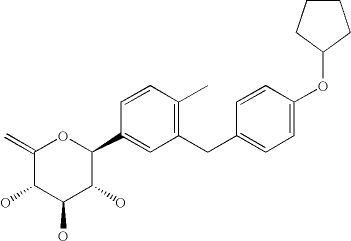 |
| (6) | 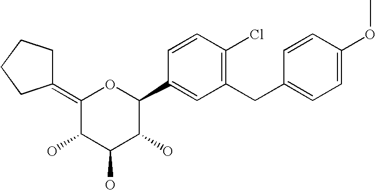 |
| (7) | 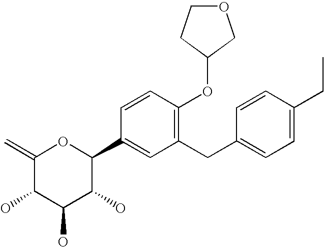 |
| (8) | 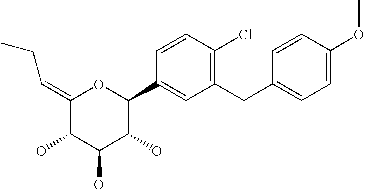 |
| (9) | 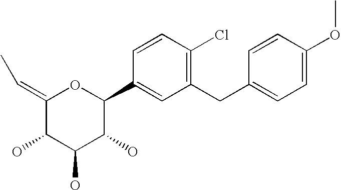 |
| (10) | 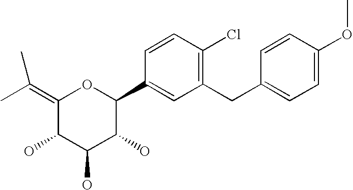 |
| (11) | 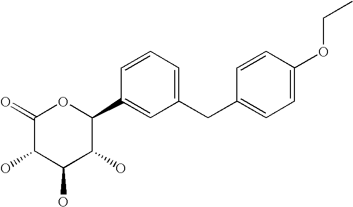 |
| (12) | 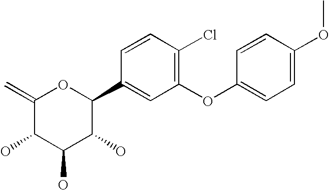 |
| (13) | 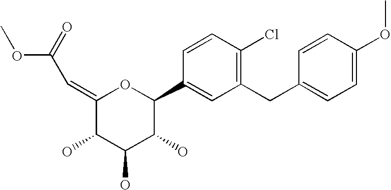 |
| (15) | 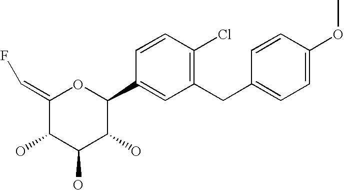 |
| (16) | 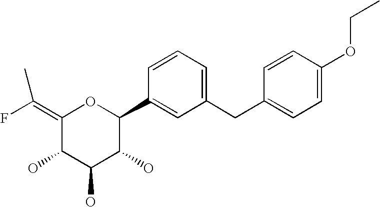 |
| (17) | 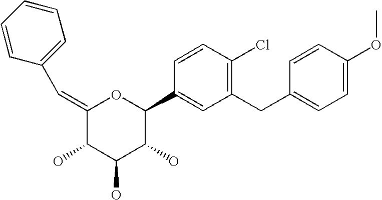 |
| (18) | 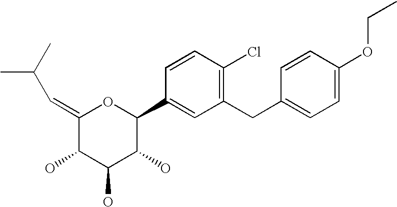 |
| (19) | 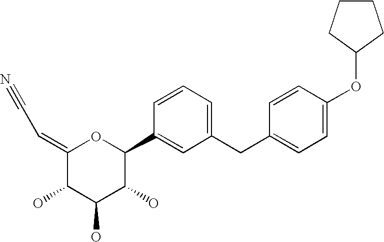 |
| (20) | 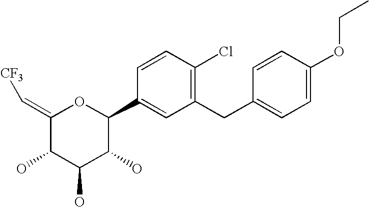 |
| (21) | 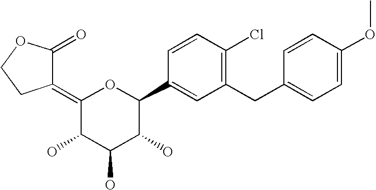 |
| (22) | 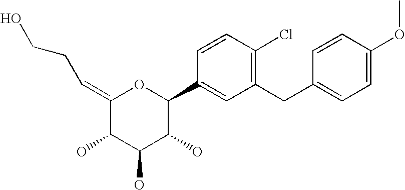 |
| (23) | 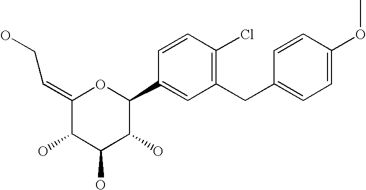 |
| (24) | 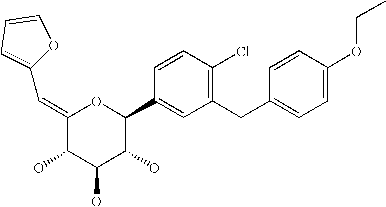 |
| (25) | 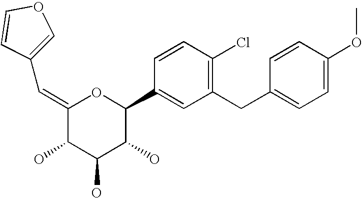 |
| (26) | 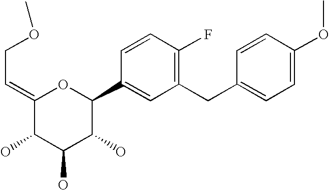 |
| (27) | 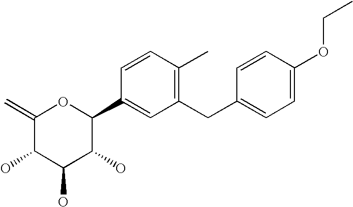 |
| (28) | 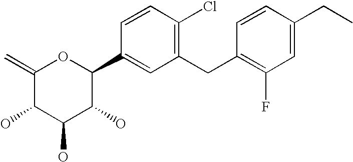 |
| (29) | 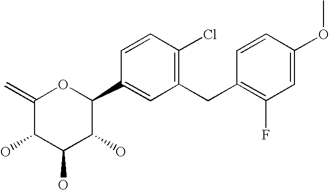 |
| (30) | 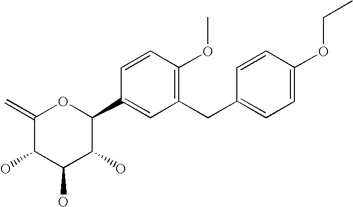 |
| (31) | 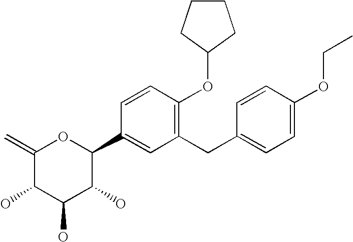 |
| (32) | 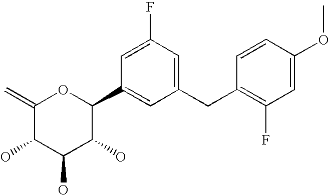 |
| (33) | 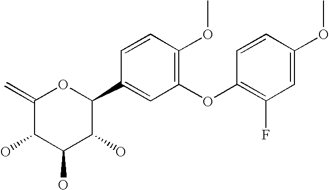 |
| (34) | 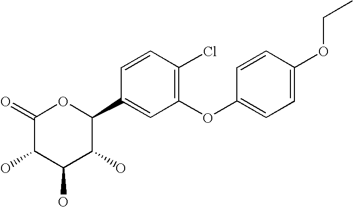 |
| (35) | 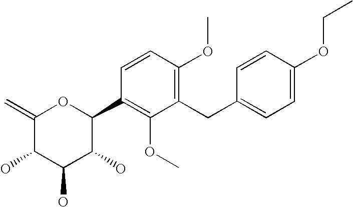 |
| (36) | 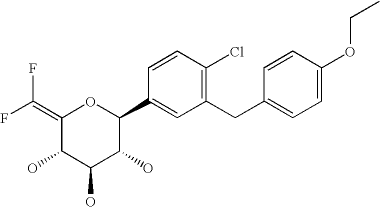 |
| (37) | 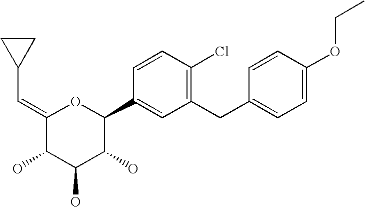 |
| (38) | 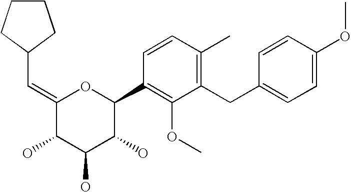 |
| (39) | 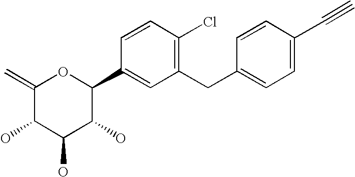 |
| (40) | 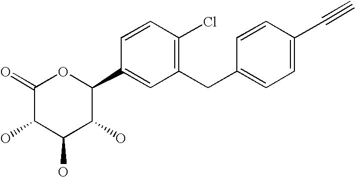 |
| (41) | 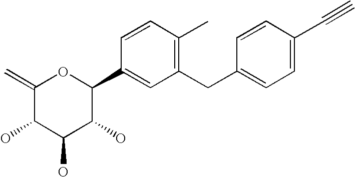 |
| (42) | 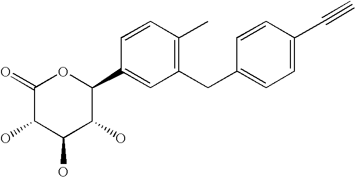 |

The following are examples of formulations in which the phrase “active substance” denotes one or more compounds according to the invention, including the salts thereof. In the case of one of the combinations with one or more other active substances the term “active substance” also includes the additional active substances.

### EXAMPLE A

Tablets containing 100 mg of active substance

| Composition: | |
|---|---|
| 1 tablet contains: | |
| active substance | 100.0 mg |
| lactose | 80.0 mg |
| corn starch | 34.0 mg |
| polyvinylpyrrolidone | 4.0 mg |
| magnesium stearate | 2.0 mg |
| | 220.0 mg |

Method of Preparation:

The active substance, lactose and starch are mixed together and uniformly moistened with an aqueous solution of the polyvinylpyrrolidone. After the moist composition has been screened (2.0 mm mesh size) and dried in a rack-type drier at 50° C. it is screened again (1.5 mm mesh size) and the lubricant is added. The finished mixture is compressed to form tablets.
Weight of tablet: 220 mg
Diameter: 10 mm, biplanar, facetted on both sides and notched on one side.

### EXAMPLE B

Tablets containing 150 mg of active substance

| Composition: | |
|---|---|
| 1 tablet contains: | |
| active substance | 150.0 mg |
| powdered lactose | 89.0 mg |
| corn starch | 40.0 mg |
| colloidal silica | 10.0 mg |
| polyvinylpyrrolidone | 10.0 mg |
| magnesium stearate | 1.0 mg |
| | 300.0 mg |

Preparation:

The active substance mixed with lactose, corn starch and silica is moistened with a 20% aqueous polyvinylpyrrolidone solution and passed through a screen with a mesh size of 1.5 mm. The granules, dried at 45° C., are passed through the same screen again and mixed with the specified amount of magnesium stearate. Tablets are pressed from the mixture.
Weight of tablet: 300 mg
die: 10 mm, flat

### EXAMPLE C

Hard Gelatine Capsules Containing 150 mg of Active Substance

| 1 capsule contains: | | |
|---|---|---|
| active substance | | 150.0 mg |
| corn starch (dried | approx. | 180.0 mg |
| lactose (powdered) | approx. | 87.0 mg |
| magnesium stearate | | 3.0 mg |
| | approx. | 420.0 mg |

Preparation:

The active substance is mixed with the excipients, passed through a screen with a mesh size of 0.75 mm and homogeneously mixed using a suitable apparatus. The finished mixture is packed into size 1 hard gelatine capsules.
Capsule filling: approx. 320 mg
Capsule shell: size 1 hard gelatine capsule.

### EXAMPLE D

Suppositories Containing 150 mg of Active Substance

| 1 suppository contains: | |
|---|---|
| active substance | 150.0 mg |
| polyethyleneglycol 1500 | 550.0 mg |
| polyethyleneglycol 6000 | 460.0 mg |
| polyoxyethylene sorbitan monostearate | 840.0 mg |
| | 2,000.0 mg |

Preparation:

After the suppository mass has been melted the active substance is homogeneously distributed therein and the melt is poured into chilled moulds.

### EXAMPLE E

Ampoules Containing 10 mg Active Substance

| Composition: | | |
|---|---|---|
| active substance | | 10.0 mg |
| 0.01 N hydrochloric acid q.s. | | |
| double-distilled water | ad | 2.0 ml |

Preparation:

The active substance is dissolved in the necessary amount of 0.01 N HCl, made isotonic with common salt, filtered sterile and transferred into 2 ml ampoules.

### EXAMPLE F

Ampoules Containing 50 mg of Active Substance

| Composition: | | |
|---|---|---|
| active substance | | 50.0 mg |
| 0.01 N hydrochloric acid q.s. | | |
| double-distilled water | ad | 10.0 ml |

Preparation:

The active substance is dissolved in the necessary amount of 0.01 N HCl, made isotonic with common salt, filtered sterile and transferred into 10 ml ampoules.

1. A D-Xylopyranosyl-substituted phenyls compound of general formula I 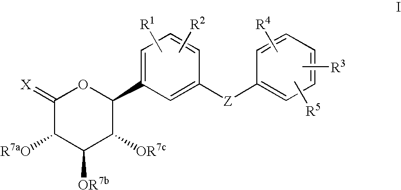
wherein
R¹ denotes hydrogen, fluorine, chlorine, bromine, C₁₋₆-alkyl, C₂₋₆-alkynyl, C₂₋₆-alkenyl, C₃₋₇-cycloalkyl, C₃₋₇-cycloalkyl-C₁₋₃-alkyl, C₅₋₇-cycloalkenyl, C₅₋₇-cycloalkenyl-C₁₋₃-alkyl, C₁₋₄-alkylcarbonyl, arylcarbonyl, heteroarylcarbonyl, aminocarbonyl, C₁₋₄-alkylaminocarbonyl, di-(C₁₋₃-alkyl)aminocarbonyl, pyrrolidin-1-ylcarbonyl, piperidin-1-ylcarbonyl, morpholin-4-ylcarbonyl, piperazin-1-ylcarbonyl, 4-(C₁₋₄-alkyl)piperazin-1-ylcarbonyl, C₁₋₄-alkoxycarbonyl, amino, C₁₋₄-alkylamino, di-(C₁₋₃-alkyl)amino, pyrrolidin-1-yl, piperidin-1-yl, morpholin-4-yl, piperazin-1-yl, 4-(C₁₋₄-alkyl)piperazin-1-yl, C₁₋₄-alkylcarbonylamino, C₁₋₆-alkyloxy, C₃₋₇-cycloalkyloxy, C₅₋₇-cycloalkenyloxy, aryloxy, C₁₋₄-alkylsulphanyl, C₁₋₄-alkylsulphinyl, C₁₋₄-alkylsulphonyl, C₃₋₇-cycloalkylsulphanyl, C₃₋₇-cycloalkylsulphinyl, C₃₋₇-cycloalkylsulphonyl, C₅₋₇-cycloalkenylsulphanyl, C₅₋₇-cycloalkenylsulphinyl, C₅₋₇-cycloalkenylsulphonyl, arylsulphanyl, arylsulphinyl, arylsulphonyl, hydroxy, cyano or nitro,
while alkyl, alkenyl, alkynyl, cycloalkyl and cycloalkenyl groups may be partly or completely fluorinated or may be mono- or disubstituted by identical or different substituents selected from chlorine, hydroxy, C₁₋₃-alkoxy and C₁₋₃-alkyl, and
in cycloalkyl and cycloalkenyl groups one or two methylene groups may be replaced independently of one another by O, S, CO, SO or SO₂, and in N-heterocycloalkyl groups a methylene group may be replaced by CO or SO₂, and
R² denotes hydrogen, fluorine, chlorine, bromine, hydroxy, C₁₋₄-alkyl, C₁₋₄-alkoxy, cyano or nitro, while alkyl groups may be mono- or polysubstituted by fluorine, or
in the event that R¹ and R² are bound to two C atoms of the phenyl ring which are adjacent to one another, R¹ and R² may be joined together in such a way that R¹ and R² together form a C₃₋₅-alkylene or C₃₋₅-alkenylene bridge, which may be partly or totally fluorinated or mono- or disubstituted by identical or different substituents selected from chlorine, hydroxy, C₁₋₃-alkoxy and C₁₋₃-alkyl and wherein one or two methylene groups may be replaced independently of one another by O, S, CO, SO, SO₂ or NR^{N},
R³ denotes hydrogen, fluorine, chlorine, bromine, C₁₋₆-alkyl, C₂₋₆-alkynyl, C₂₋₆-alkenyl, C₃₋₇-cycloalkyl, C₃₋₇-cycloalkyl-C₁₋₃-alkyl, C₅₋₇-cycloalkenyl, C₅₋₇-cycloalkenyl-C₁₋₃-alkyl, aryl, heteroaryl, C₁₋₄-alkylcarbonyl, arylcarbonyl, heteroarylcarbonyl, aminocarbonyl, C₁₋₄-alkylaminocarbonyl, di-(C₁₋₃-alkyl)aminocarbonyl, pyrrolidin-1-ylcarbonyl, piperidin-1-ylcarbonyl, morpholin-4-ylcarbonyl, piperazin-1-ylcarbonyl, 4-(C₁₋₄-alkyl)piperazin-1-ylcarbonyl, hydroxycarbonyl, C₁₋₄-alkoxycarbonyl, C₁₋₄-alkylamino, di-(C₁₋₃-alkyl)amino, pyrrolidin-1-yl, piperidin-1-yl, morpholin-4-yl, piperazin-1-yl, 4-(C₁₋₄-alkyl)piperazin-1-yl, C₄-alkylcarbonylamino, arylcarbonylamino, heteroarylcarbonylamino, C₁₋₄-alkylsulphonylamino, arylsulphonylamino, C₁₋₆-alkoxy, C₃₋₇-cycloalkyloxy, C₅₋₇-cycloalkenyloxy, aryloxy, heteroaryloxy, C₁₋₄-alkylsulphanyl, C₁₋₄-alkylsulphinyl, C₁₋₄-alkylsulphonyl, C₃₋₇-cycloalkyl-sulphanyl, C₃₋₇-cycloalkylsulphinyl, C₃₋₇-cycloalkylsulphonyl, C₅₋₇-cycloalkenylsulphanyl, C₅₋₇-cycloalkenylsulphinyl, C₅₋₇-cycloalkenylsulphonyl, arylsulphanyl, arylsulphinyl, arylsulphonyl, amino, hydroxy, cyano or nitro,
while alkyl, alkenyl, alkynyl, cycloalkyl and cycloalkenyl groups may be partly or totally fluorinated or mono- or disubstituted by identical or different substituents selected from chlorine, hydroxy, C₁₋₃-alkoxy and C₁₋₃-alkyl, and
in cycloalkyl and cycloalkenyl groups one or two methylene groups may be replaced independently of one another by O, S, CO, SO or SO₂, and
in N-heterocycloalkyl groups a methylene group may be replaced by CO or SO₂, and
R⁴ denotes hydrogen, fluorine, chlorine, bromine, iodine, cyano, nitro, C₁₋₃-alkyl, C₁₋₃-alkoxy or methyl or methoxy substituted by 1 to 3 fluorine atoms, or
in the event that R³ and R⁴ are bound to two C atoms of the phenyl ring which are adjacent to one another, R³ and R⁴ may be joined together in such a way that R³ and R⁴ together form a C₃₋₅-alkylene or C₃₋₅-alkenylene bridge, which may be partly or totally fluorinated or mono- or disubstituted by identical or different substituents selected from chlorine, hydroxy, C₁₋₃-alkoxy and C₁₋₃-alkyl and wherein one or two methylene groups may be replaced independently of one another by O, S, CO, SO, SO₂ or NR^{N},
R⁵ denotes hydrogen, fluorine, chlorine, bromine, iodine, cyano, nitro, C₁₋₃-alkyl, C₁₋₃-alkoxy or methyl or methoxy substituted by 1 to 3 fluorine atoms, and
R^{N} independently of one another denote H or C₁₋₄-alkyl,
L are selected independently of one another from among fluorine, chlorine, bromine, iodine, C₁₋₃-alkyl, difluoromethyl, trifluoromethyl, C₁₋₃-alkoxy, difluoromethoxy, trifluoromethoxy and cyano,
R^{7a}, R^{7b}
R^{7c} independently of one another have a meaning selected from among hydrogen, (C₁₋₁₈-alkyl)carbonyl, (C₁₋₁₈-alkyl)oxycarbonyl, arylcarbonyl and aryl-(C₁₋₃-alkyl)-carbonyl,
X denotes oxygen, or
methylidene, fluoromethylidene, C₁₋₆-alkyl-methylidene, C₂₋₆-alkenyl-methylidene, C₂₋₆-alkynyl-methylidene, C₃₋₇-cycloalkyl-methylidene, C₅₋₇-cycloalkenyl-methylidene, C₃₋₇-cycloalkylidene, C₅₋₇-cycloalkenylidene, C₃₋₇-cycloalkyl-C₁₋₃-alkyl-methylidene, C₅₋₇-cycloalkenyl-C₁₋₃-alkyl-methylidene, arylmethylidene, heteroarylmethylidene, aryl-C₁₋₃-alkyl-methylidene or heteroaryl-C₁₋₃-alkyl-methylidene,
while alkyl, alkenyl, alkynyl, cycloalkyl and cycloalkenyl groups may be partly or totally fluorinated or mono- or disubstituted by identical or different substituents selected from chlorine, cyano, hydroxy, C₁₋₃-alkoxy and C₁₋₃-alkyl, and
the above-mentioned unsubstituted methylidene group or the above-mentioned monosubstituted methylidene groups may additionally be monosubstituted by fluorine, chlorine, C₁₋₃-alkyl, cyano or nitro, and
in cycloalkyl, cycloalkenyl, cycloalkylidene and cycloalkenylidene groups one or two methylene groups may independently of one another be replaced by O, S, CO, SO, SO₂ or NR^{N}, or
X denotes a group according to partial formula 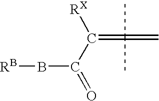
wherein
R^{X} denotes hydrogen, fluorine, chlorine, cyano, trifluoromethyl or C₁₋₃-alkyl,
B denotes a single bond, —O— or —NR^{N}—,
R^{B} denotes hydrogen, C₁₋₆-alkyl, C₃₋₆-alkenyl, C₃₋₆-alkynyl, C₃₋₇-cycloalkyl, C₅₋₇-cycloalkenyl, C₃₋₇-cycloalkyl-C₁₋₃-alkyl, C₅₋₇-cycloalkenyl-C₁₋₃-alkyl, aryl, heteroaryl, aryl-C₁₋₃-alkyl or heteroaryl-C₁₋₃-alkyl,
while alkyl, cycloalkyl and cycloalkenyl groups may be partly or completely fluorinated or mono- or disubstituted by identical or different substituents selected from chlorine, cyano, hydroxy, C₁₋₃-alkoxy and C₁₋₃-alkyl, or
R^{B} and B are joined together, forming a heterocyclic ring selected from pyrrolidine, morpholine, piperidine, piperazine and 4-(C₁₋₄-alkyl)-piperazine, the heterocyclic ring being bound to the C═O— group via the imino group,
Z denotes oxygen, methylene, dimethylmethylene, difluoromethylene or carbonyl;
while the term aryl groups used in the definition of the above groups denotes phenyl or naphthyl groups, which may be mono- or disubstituted independently of one another by identical or different groups L; and
the term heteroaryl groups used in the definition of the above-mentioned groups denotes a pyrrolyl, furanyl, thienyl, pyridyl, indolyl, benzofliranyl, benzothio-phenyl, quinolinyl or isoquinolinyl group,
or a pyrrolyl, furanyl, thienyl, imidazolyl or pyridyl group, wherein one or two methyne groups are replaced by nitrogen atoms,
or an indolyl, benzofuranyl, benzothiophenyl, quinolinyl or isoquinolinyl group, wherein one to three methyne groups are replaced by nitrogen atoms,
while the above-mentioned heteroaryl groups may be mono- or disubstituted independently of one another by identical or different groups L;
while by the N-heterocycloalkyl group mentioned in the definition of the above-mentioned groups is meant a saturated carbocyclic ring which comprises an imino group in the ring, which may comprise another optionally substituted imino group or an O or S atom in the ring, and
unless otherwise stated the above-mentioned alkyl groups may be straight-chain or branched,
the tautomers, the stereoisomers, the mixtures thereof and the salts thereof, particularly the physiologically acceptable salts thereof.

2. A D-Xylopyranosyl-substituted phenyl according to claim 1, characterised by the formula I.2 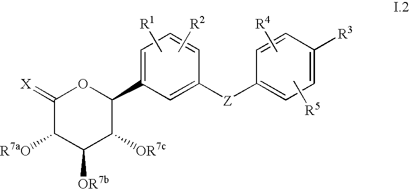
wherein R¹ to R⁵, X, Z, R^{7a}, R^{7b}, R^{7c} have the meanings according to claim 1.

3. A D-Xylopyranosyl-substituted phenyl according to claim 1, characterised by the formula I.2c 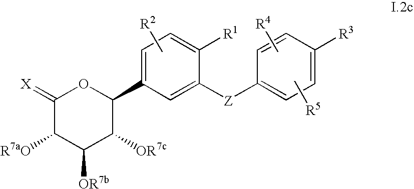
wherein R¹ to R⁵, X, Z, R^{7a}, R^{7b}, R^{7c} have the meanings according to claim 1.

4. A D-Xylopyranosyl-substituted phenyl according to claim 1 characterised in that
R¹ denotes hydrogen, fluorine, chlorine, bromine, C₁₋₆-alkyl, C₂₋₆-alkynyl, C₂₋₆-alkenyl, C₃₋₇-cycloalkyl, C₅₋₇-cycloalkenyl, C₁₋₆-alkyloxy, C₃₋₇-cycloalkyloxy or cyano, while in cycloalkyl and cycloalkenyl groups one or two methylene units may be replaced independently of one another by O or CO and alkyl, alkenyl and alkynyl groups may be partly or completely fluorinated.

5. A D-Xylopyranosyl-substituted phenyl according to claim 1, characterised in that
R³ denotes C₁₋₆-alkyl, C₂₋₆-alkynyl, C₁₋₄-alkyloxy, C₃₋₇-cycloalkyl, C₃₋₇-cycloalkyloxy or hydroxy, while in the cycloalkyl groups one or two methylene units may be replaced independently of one another by O or CO and alkyl groups may be partly or completely fluorinated.

6. A D-Xylopyranosyl-substituted phenyl according to claims 1, characterised in that X denotes oxygen, methylidene, fluoromethylidene, C₁₋₆-alkyl-methylidene, C₂₋₆-alkynyl-methylidene, C₂₋₆-alkenyl-methylidene, C₃₋₇-cycloalkyl-methylidene or C₃₋₇-cycloalkylidene,
while the above-mentioned alkyl, alkenyl and alkynyl groups may be partly or completely fluorinated and may be mono- or disubstituted independently of one another by substituents selected from chlorine, hydroxy, C₁₋₃-alkoxy and C₁₋₃-alkyl, and
the above-mentioned unsubstituted methylidene group or the above-mentioned monosubstituted methylidene groups may additionally be monosubstituted by fluorine, chlorine, C₁₋₃-alkyl or cyano, and
in a cycloalkylidene group a methylene group may be replaced by O, S or NR^{N} or an ethylene group may be replaced by —NR^{N}—CO—, —CO—NR^{N}—, —O—CO— or —CO—O—; or
X preferably denotes a group according to partial formula T 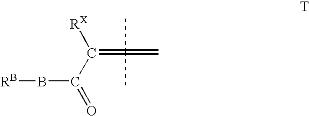
wherein
R^{X} denotes hydrogen, fluorine, cyano, trifluoromethyl or C₁₋₃-alkyl,
B denotes a single bond, —O— or —NR^{N}—,
R^{B} denotes C₁₋₆-alkyl, C₃₋₇-cycloalkyl, C₅₋₇-cycloalkenyl, C₃₋₇-cycloalkyl-C₁₋₃-alkyl, C₅₋₇-cycloalkenyl-C₁₋₃-alkyl, aryl, heteroaryl, aryl-C₁₋₃-alkyl or heteroaryl-C₁₋₃-alkyl,
while alkyl, cycloalkyl and cycloalkenyl groups may be partly or completely fluorinated or mono- or disubstituted by identical or different substituents selected from cyano, hydroxy, C₁₋₃-alkoxy and C₁₋₃-alkyl, or
R^{B} and B are joined together, forming a heterocyclic ring selected from pyrrolidine, morpholine, piperidine, piperazine and 4-(C₁₋₄-alkyl)-piperazine, the heterocyclic ring being bound to the C═O— group via the imino group and
R^{N} is defined according to claim 1.

7. A D-Xylopyranosyl-substituted phenyl according to claim 1 characterised in that X denotes oxygen.

8. D-Xylopyranosyl-substituted phenyls according to claim 1 characterised in that X denotes methylidene, fluoromethylidene, C₁₋₆-alkyl-methylidene, C₂₋₆-alkynyl-methylidene, C₂₋₆-alkenyl-methylidene, C₃₋₇-cycloalkyl-methylidene or C₃₋₇-cycloalkylidene,
while the above-mentioned alkyl, alkenyl and alkynyl groups may be partly or completely fluorinated and may be mono- or disubstituted independently of one another by substituents selected from chlorine, hydroxy, C₁₋₃-alkoxy and C₁₋₃-alkyl, and
the above-mentioned unsubstituted methylidene group or the above-mentioned monosubstituted methylidene groups may additionally be monosubstituted by fluorine, chlorine, C₁₋₃-alkyl or cyano, and
in a cycloalkylidene group a methylene group may be replaced by O, S or NR^{N} or an ethylene group may be replaced by —NR^{N}—CO—, —CO—NR^{N}—, —O—CO— or —CO—O—, and
R^{N} is defined according to claim 1.

9. A D-Xylopyranosyl-substituted phenyls according to claim 1, characterised in that X is a group according to partial formula T 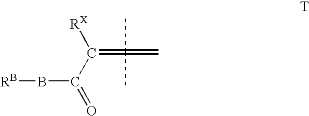
wherein
R^{X} denotes hydrogen, fluorine, cyano, trifluoromethyl or C₁₋₃-alkyl,
B denotes a single bond, —O— or —NR^{N}—,
R^{B} denotes C₁₋₆-alkyl, C₃₋₇-cycloalkyl, C₁₋₇-cycloalkenyl, C₃₋₇-cycloalkyl-C₁₋₃-alkyl, C₅₋₇-cycloalkenyl-C₁₋₃-alkyl, aryl, heteroaryl, aryl-C₁₋₃-alkyl or heteroaryl-C₁₋₃-alkyl,
while alkyl, cycloalkyl and cycloalkenyl groups may be partly or completely fluorinated or mono- or disubstituted by identical or different substituents selected from cyano, hydroxy, C₁₋₃-alkoxy and C₁₋₃-alkyl, or
R^{B} and B are joined together, forming a heterocyclic ring selected from pyrrolidine, morpholine, piperidine, piperazine and 4-(C₁₋₄-alkyl)-piperazine, the heterocyclic ring being bound to the C═O— group via the imino group, and
R^{N} is defined according to claim 1.

10. A D-Xylopyranosyl-substituted phenyls according to claims 1, characterised in that
R² denotes hydrogen, fluorine, hydroxy, methoxy, ethoxy or methyl.

11. A D-Xylopyranosyl-substituted phenyl according to claim 1, characterised in that
R⁴ and R⁵ independently of one another represent hydrogen or fluorine.

12. A D-Xylopyranosyl-substituted phenyl according to claim 1 characterised in that
Z denotes oxygen or methylene.

13. A D-Xylopyranosyl-substituted phenyl according to claim 1 characterised in that
R^{7a}, R^{7b}, R^{7c} independently of one another represent hydrogen, (C₁₋₆-alkyl)oxycarbonyl, (C₁₋₈-alkyl)carbonyl or benzoyl, preferably hydrogen.

14. Physiologically acceptable salts of the compounds according to claim 1 with inorganic or organic acids.

15. A pharmaceutical composition comprised of a compound according to claim 1 or a physiologically acceptable salt thereof as a pharmaceutical composition.

16. A pharmaceutical composition comprised of a compound according to claim 1 or a physiologically acceptable salt thereof optionally together with one or more inert carriers and/or diluents.

17. A method of treating or preventing diseases or conditions which can be influenced by inhibiting the sodium-dependent glucose cotransporter SGLT said method comprised of the steps of administering to a patient in need thereof a therapeutically effective amount of a compound according to claim 1.

18. A method of treating or preventing metabolic disorders, said method comprised of the step of administering to a patient in need thereof a therapeutically effective amount of a compound according to claim 1 or a physiologically acceptable salt thereof.

19. The method of claim 18 wherein the metabolic disorder is selected from the group consisting of type 1 and type 2 diabetes mellitus, complications of diabetes, metabolic acidosis or ketosis, reactive hypoglycaemia, hyperinsulinaemia, glucose metabolic disorder, insulin resistance, metabolic syndrome, dyslipidaemias of different origins, atherosclerosis and related diseases, obesity, high blood pressure, chronic heart failure, oedema and hyperuricaemia.

20. A method of inhibiting the sodium-dependent glucose contransporter SGLT in a patient in need thereof said method comprised of the step of administering a therapeutically effective amount of a compound according to claim 1 or a physiologically acceptable salt thereof.

21. A method of preventing the degeneration of pancreatic beta cells and/or for improving and/or restoring the functionality of pancreatic beta cells in a patient in need thereof said method comprised of the step of administering a therapeutically effective amount of a compound according to claim 1 or a physiologically acceptable salt thereof.

22. A method of treating hypertension said method comprised on the step of administering to a patient in need thereof a therapeutically effective amount of a compound according to claim 1 or a physiologically acceptable salt thereof.

23. A method of treating conditions requiring a diuretic method comprised on the step of administering to a patient in need thereof a therapeutically effective amount of a compound according to claim 1 or a physiologically acceptable salt thereof.

24. Process for preparing a pharmaceutical composition according to claim 1, characterised in that said compound or a physiologically acceptable salt thereof is incorporated in one or more inert carriers and/or diluents by a non-chemical method.
